SEQUENCE LISTING (6 sequences)

SEQ ID NO: 1 - protein, 491 aa, Lassa virus
MGQIVTFFQEVPHVIEEVMNIVLIALSVLAVLKGLYNFATCGLVGLVTFLLLCGRSCTTSLYKGVYELQTLELNMETLNMTMPLSCTKNNSHHYIMVGNETGLELTLTNTSIINHKFCNLSDAHKKNLYDHALMSIISTFHLSIPNFNQYEAMSCDFNGGKISVQYNLSHSYAGDAANHCGTVANGVLQTFMRMAWGGSYIALDSGRGNWDCIMTSYQYLIIQNTTWEDHCQFSRPSPIGYLGLLSQRTRDIYISRRLLGTFTWTLSDSEGKDTPGGYCLTRWMLIEAELKCFGNTAVAKCNEKHDEEFCDMLRLFDFNKQAIQRLKAEAQMSIQLINKAVNALINDQLIMKNHLRDIMGIPYCNYSKYWYLNHTTTGRTSLPKCWLVSNGSYLNETHFSDDIEQQADNMITEMLQKEYMERQGKTPLGLVDLFVFSTSFYLISIFLHLVKIPTHRHIVGKSCPKPHRLNHMGICSCGLYKQPGVPVKWKR

SEQ ID NO: 2 - protein, 468 aa, Artificial sequence
  Engineered LASV GPC protein
MGQIVTFFQEVPHVIEEVMNIVLIALSVLAVLKGLYNFATCGLVGLVTFLLLCGRSCTTSLYKGVYELQTLELNMETLNMTMPLSCTKNNSHHYIMVGNETGLELTLTNTSIINHKFCNLSDAHKKNLYDHALMSIISTFHLSIPNFNQYEAMSCDFNGGKISVQYNLSHSYAGDAANHCGTVANGVLQTFMRMAWGGSYIALDSGGCGNWDCIMTSYQYLIIQNTTWEDHCQFSRPSPIGYLGLLSQRTRDIYISRRRRGTFTWTLSDSEGKDTPGGYCLTRWMLIEAELKCFGNTAVAKCNEKHDEEFCDMLRLFDFNKQAIQRCKAPAQMSIQLINKAVNALINDQLIMKNHLRDIMGIPYCNYSKYWYLNHTTTGRTSLPKCWLVSNGSYLNETHFSDDIEQQADNMITEMLQKEGGGYIPEAPRDGQAYVRKDGEWVLLSTFLGGGSGAHIVMVDAYKPTKGG

SEQ ID NO: 3 - protein, 492 aa, Artificial sequence
  Engineered LASV GPC protein
MGQIVTFFQEVPHVIEEVMNIVLIALSVLAVLKGLYNFATCGLVGLVTFLLLCGRSCTTSLYKGVYELQTLELNMETLNMTMPLSCTKNNSHHYIMVGNETGLELTLTNTSIINHKFCNLSDAHKKNLYDHALMSIISTFHLSIPNFNQYEAMSCDFNGGKISVQYNLSHSYAGDAANHCGTVANGVLQTFMRMAWGGSYIALDSGGCGNWDCIMTSYQYLIIQNTTWEDHCQFSRPSPIGYLGLLSQRTRDIYISRRRRGTFTWTLSDSEGKDTPGGYCLTRWMLIEAELKCFGNTAVAKCNEKHDEEFCDMLRLFDFNKQAIQRCKAEAQMSIQLINKAVNALINDQLIMKNHLRDIMGIPYCNYSKYWYLNHTTTGRTSLPKCWLVSNGSYLNETHFSDDIEQQADNMITEMLQKEYMERQGKTPLGLVDLFVFSTSFYLISIFLHLVKIPTHRHIVGKSCPKPHRLNHMGICSCGLYKQPGVPVKWKR

SEQ ID NO: 4 - protein, 26 aa, Artificial sequence
  T4 Fibritin trimerization domain
GYIPEAPRDGQAYVRKDGEWVLLSTF

SEQ ID NO: 5 - protein, 338 aa, Artificial sequence
  Engineered LASV GPC protein
PYGWEYAAHPLCEVEVLSDENEVVKWGLRKSLPLIELRATFTLLWELDNLECGKPNVDLSSLEETVRKVAEFEDEVIFRGCEKSGVKGLLSFEERKIECGSTPKDLLEAIVRALSIFSKDGIEGPYTLVINTDRWINFLKEEAGHYPLEKRVEECLRGGKIITTPRIEDALVVSERGGDFKLILGQDLSIGYEDREKDAVRLFITETFTMLLKFGSGSGSVTTLSGLSGEQGPSGDMTTEEDSATHIKFSKRDEDGRELAGATMELRDSSGKTISTWISDGHVKDFYLYPGKYTFVETAAPDGYEVATAITFTVNEQGQVTVNGEATKGDAHTGSSGS

SEQ ID NO: 6 - protein, 394 aa, Artificial sequence
  Engineered LASV GPC protein
MEFLKRSFAPLTEKQWQEIDNRAREIFKTQLYGRKFVDVEGGGGGHHHHHHGGGGGPYGWEYAAHPLCEVEVLSDENEVVKWGLRKSLPLIELRATFTLLWELDNLECGKPNVDLSSLEETVRKVAEFEDEVIFRGCEKSGVKGLLSFEERKIECGSTPKDLLEAIVRALSIFSKDGIEGPYTLVINTDRWINFLKEEAGHYPLEKRVEECLRGGKIITTPRIEDALVVSERGGDFKLILGQDLSIGYEDREKDAVRLFITETFTMLLKFGSGSGSVTTLSGLSGEQGPSGDMTTEEDSATHIKFSKRDEDGRELAGATMELRDSSGKTISTWISDGHVKDFYLYPGKYTFVETAAPDGYEVATAITFTVNEQGQVTVNGEATKGDAHTGSSGS

(19) United States
(12) Patent Application Publication
(10) Pub. No.: US 2024/0228549 A1
(43) Pub. Date: Jul. 11, 2024

(54) PREFUSION-STABILIZED LASSA VIRUS GLYCOPROTEIN COMPLEX AND ITS USE

(71) Applicant: The U.S.A., as represented by the Secretary, Department of Health and Human Services, Bethesda, MD (US)

(72) Inventors: Peter Kwong, Washington, DC (US); Li Ou, Potomac, MD (US); Jason J. Gorman, Silver Spring, MD (US)

(73) Assignee: The U.S.A., as represented by the Secretary, Department of Health and Human Services, Bethesda, MD (US)

(21) Appl. No.: 18/557,396

(22) PCT Filed: Apr. 29, 2022
(86) PCT No.: PCT/US2022/027136

§ 371 Date: Oct. 26, 2023

Related U.S. Application Data
(60) Provisional application No. 63/181,519, filed on Apr. 29, 2021.
(60) Provisional application No. 63/329,886, filed on Apr. 12, 2022.

(51) Int. Cl. C07K 14/005; A61K 39/00; A61K 39/12; A61P 37/02

(52) U.S. Cl. CPC C07K 14/005; A61K 39/12; A61P 37/02; A61K 2039/55555; C07K 2319/735; C12N 2760/10022; C12N 2760/10034

(57) ABSTRACT

Immunogens comprising a recombinant Lassa virus glycoprotein complex ectodomain trimer stabilized in a prefusion closed conformation, as well as methods of their use and production are disclosed. In several embodiments, the immunogen can be used to elicit an immune response to Lassa virus in a subject.

### CROSS REFERENCE TO RELATED APPLICATION

This application claims priority to U.S. Provisional Application No. 63/329,886, filed Apr. 12, 2022, and U.S. Provisional Application No. 63/181,519, filed Apr. 29, 2021, each which is incorporated by reference in its entirety.

### FIELD

This disclosure relates to a recombinant Lassa virus Glycoprotein Complex that is stabilized in a prefusion conformation, and its use as an immunogen.

### BACKGROUND

Lassa virus (LASV), an Old World arenavirus that causes the acute viral hemorrhagic illness Lassa fever, is highly prevalent in West Africa, infecting 100,000 to 300,000 individuals each year, with approximately 5,000 deaths. The LASV outbreak in Nigeria in 2018 had more than 300 confirmed cases, with a case fatality rate of approximately 25%. No licensed vaccine is available for the prevention of Lassa fever, and the only treatment is ribavirin, a broad spectrum antiviral drug. With the growing threat of spreading globally, LASV has been included on the priority pathogen list for the World Health Organization's R&D Blueprint for Action to Prevent Epidemics in an urgent effort to develop effective vaccines.

The majority of LASV vaccine development efforts have focused on the glycoprotein complex (GPC) present on the virion envelope. However, there has been very limited success due to the highly glycosylated GPC inducing only a weak and inconsistent immune response in both natural infection and vaccination.

### SUMMARY

Provided herein is a recombinant Lassa virus (LASV) Glycoprotein Complex (GPC) ectodomain trimer stabilized in a prefusion conformation, which has utility, for example, as an immunogen for elicitation of an immune response to LASV GPC in a subject. Stabilization in the prefusion conformation is accomplished by one or more amino acid substitutions in protomers of the trimer. In some embodiments, the amino acid substitutions comprising cysteine substitutions at GPC positions 207 and 326 (such as R207C and L326C) that form a non-natural interprotomer disulfide bond. In some embodiments, the protomers further comprising a glycine insertion between GPC positions 205 and 206. In some embodiments, the protomers of the LASV GPC ectodomain are C-terminally fused to a trimerization domain, such as a T4 fibritin trimerization domain.

Nanoparticles including a disclosed LASV GPC ectodomain trimer are also provided. In some embodiments, the nanoparticles are self-assembling encapsulin nanoparticles linked to the LASV GPC ectodomain trimer using the spytag:spycatcher isopeptide bond conjugation system.

Virus-like particles including disclosed LASV GPC ectodomain trimer are also provided.

Nucleic acid molecules encoding the disclosed recombinant LASV GPC ectodomain trimers are also provided. In some embodiments, the nucleic acid molecule can encode a precursor protein of a protomer of a disclosed recombinant LASV GPC trimer. Expression vectors (such as an inactivated or attenuated viral vector) including the nucleic acid molecules are also provided.

Immunogenic compositions including one or more of the disclosed recombinant LASV GPC ectodomain trimers and/or nanoparticles, are also provided. The composition may be contained in a unit dosage form. The composition can further include an adjuvant.

Methods of eliciting an immune response to LASV GPC in a subject are disclosed, as are methods of treating, inhibiting or preventing an LASV infection in a subject. In such methods a subject, such as a human subject, is administered an effective amount of a disclosed recombinant LASV GPC ectodomain trimer to elicit the immune response. The subject can be, for example, a human subject at risk of or having an LASV infection.

The foregoing and other objects and features of the disclosure will become more apparent from the following detailed description, which proceeds with reference to the accompanying figures.

### BRIEF DESCRIPTION OF THE DRAWINGS

FIGS. 1A-1F: Design and characterization of stabilized soluble LASV GPC trimer. (FIG. 1A) Structure-based design of stabilized soluble Lassa virus GPC trimer. An inter-protomer disulfide (DS) bond linked GP1 of one protomer to GP2 of a neighboring protomer. A foldon domain was appended to the C-terminus of GP2. The right panel shows a zoom-in view around the inter-protomer DS with the mutations to create the DS marked by spheres and labeled. In the template structure GPCysR4 (PDB: 5VK2), cysteines introduced by R207C/G360C substitutions form an intra-protomer disulfide bond. The prefusion GPC designs described herein do not include this intra-protomer disulfide and residue 360 is glycine, which is the residue present at this position in native sequence. The two front protomers are shown as ribbons. The protomer in the back is shown as a white surface. (FIG. 1B) SDS-PAGE of stabilized LASV trimer under non-reducing and reducing conditions. A high molecular weight band three times the molecular weight of the monomeric form was observed in the non-reducing condition. (FIG. 1C) Binding affinity of the stabilized LASV GPC trimer toward Fabs of four groups of human LASV-neutralizing antibodies: GP1-A, GPC-A, GPC-B, and GPC-C. (FIG. 1D) Negative-stain electron microscopy (EM) images of the stabilized LASV GPC trimer alone and in complex with 4 groups of human neutralizing Fabs. Representative top view and side view are shown. (FIG. 1E) Cryo-EM structure of the stabilized LASV GPC at 4 Å revealed the trimeric association of the protein. (FIG. 1F) Physical properties of the stabilized LASV GPC trimer. Stability of the stabilized trimer was assessed as fractional binding reactivity to 37.7H after treatments under various temperatures, pH, osmolarity changes and freeze-thaw cycles. Triplicate measurements were made, and results are represented as mean±SEM. The dotted line shows the antibody reactivity of the trimer prior to physical stress.

FIGS. 2A-2D: Antigenic screening of designed prefusion-stabilized Lassa GPC constructs. 164 variants were designed, which were screened for binding to the quaternary-specific GPC antibody 37.7H under physiological conditions, at lower pH (5.5) and at higher temperature (56° C.). Numbers indicate ELISA results (OD₄₅₀) for recognition by 37.7H. Controls are indicated with green background. Highlighted in blue in the “Construct description” column are the two designs, inter-protomer disulfide and foldon fusion, that were combined in the final construct for detailed characterization in this study after evaluation of their behavior at 1 liter expression and the ability to combine them.

FIGS. 3A-3C: Construct, yield, and purification profile of stabilized soluble Lassa virus GPC trimer. (FIG. 3A) Schematic showing the design of GPCysR4 (top) and stabilized soluble Lassa virus GPC trimer (bottom). The original C207-C360 intra-protomer disulfide in GPCysR4 was abolished by mutating C360G. A new inter-protomer disulfide was created between C207 and L326C. Insertion of G206A allowed optimal geometry for disulfide bond formation. A T4-fibritin (foldon) trimerization domain was also introduced at the C-terminus to fix the base of the trimer. (FIG. 3B) Protein yield of the stabilized soluble GPC trimer following nickel-affinity (Ni), streptavidin-affinity (Strep), and size exclusion (SEC) purification. (FIG. 3C) SEC profile of the stabilized soluble GPC trimer on Superdex 200 16/600 column.

FIG. 4 depicts generation of encapsulin nanoparticles linked to prefusion LASV GPC ectodomain trimer using the spytag:spycatcher system. The nanoparticles include prefusion LASV GPC ectodomain trimers with C-terminal fusion to T4 fibritin trimerization domain and spytag (GPC^{spyT}) linked to self-assembled encapsulin nanoparticles composed of subunits fused to spycatcher (EN^{spyC}).

FIG. 5 shows cryo-EM assessment of the encapsulin nanoparticles linked to prefusion LASV GPC ectodomain trimer using the spytag:spycatcher system

FIG. 6 shows results of ELISA assays for immune sera from mice immunized with prefusion LASV GPC ectodomain trimer binding to the prefusion LASV GPC ectodomain trimer on the ELISA plate.

FIG. 7 shows results of Octet binding response assays for immune sera from guinea pigs immunized with prefusion LASV GPC ectodomain trimer followed by boost with the encapsulin nanoparticles linked to prefusion LASV GPC ectodomain trimer. Sera binding to the prefusion LASV GPC ectodomain trimer was assessed.

FIG. 8 shows results of Lassa pseudovirus neutralization assays of immune sera taken from guinea pigs immunized with prefusion LASV GPC ectodomain trimer followed by boost with the encapsulin nanoparticles linked to prefusion LASV GPC ectodomain trimer.

### SEQUENCE LISTING

The nucleic and amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. The Sequence Listing is submitted as an ASCII text file in the form of the file named “Sequence.txt” (˜20 kb), which was created on Apr. 29, 2022, which is incorporated by reference herein. In the accompanying sequence listing:

wild type LASV GPC Josiah
SEQ ID NO: 1
MGQIVTFFQEVPHVIEEVMNIVLIALSVLAVLKGLYNFATCGLVGLVTF
LLLCGRSCTTSLYKGVYELQTLELNMETLNMTMPLSCTKNNSHHYIMVG
NETGLELTLTNTSIINHKFCNLSDAHKKNLYDHALMSIISTFHLSIPNF
NQYEAMSCDFNGGKISVQYNLSHSYAGDAANHCGTVANGVLQTFMRMAW
GGSYIALDSGRGNWDCIMTSYQYLIIQNTTWEDHCQFSRPSPIGYLGLL
SQRTRDIYISRRLLGTFTWTLSDSEGKDTPGGYCLTRWMLIEAELKCFG
NTAVAKCNEKHDEEFCDMLRLFDFNKQAIQRLKAEAQMSIQLINKAVNA
LINDQLIMKNHLRDIMGIPYCNYSKYWYLNHTTTGRTSLPKCWLVSNGS
YLNETHFSDDIEQQADNMITEMLQKEYMERQGKTPLGLVDLFVFSTSFY
LISIFLHLVKIPTHRHIVGKSCPKPHRLNHMGICSCGLYKQPGVPVKWK
R
LASV GPC ectodomain, R207GC/L326C, L258R/L259R,
foldon, spytag
SEQ ID NO: 2
MGQIVTFFQEVPHVIEEVMNIVLIALSVLAVLKGLYNFATCGLVGLVTF
LLLCGRSCTTSLYKGVYELQTLELNMETLNMTMPLSCTKNNSHHYIMVG
NETGLELTLTNTSIINHKFCNLSDAHKKNLYDHALMSIISTFHLSIPNF
NQYEAMSCDFNGGKISVQYNLSHSYAGDAANHCGTVANGVLQTFMRMAW
GGSYIALDSGGCGNWDCIMTSYQYLIIQNTTWEDHCQFSRPSPIGYLGL
LSQRTRDIYISRRRRGTFTWTLSDSEGKDTPGGYCLTRWMLIEAELKCF
GNTAVAKCNEKHDEEFCDMLRLFDFNKQAIQRCKAPAQMSIQLINKAVN
ALINDQLIMKNHLRDIMGIPYCNYSKYWYLNHTTTGRTSLPKCWLVSNG
SYLNETHFSDDIEQQADNMITEMLQKEGGGYIPEAPRDGQAYVRKDGEW
VLLSTFLGGGSGAHIVMVDAYKPTKGG
LASV GPC, R207GC/L326C, L258R/L259R
SEQ ID NO: 3
MGQIVTFFQEVPHVIEEVMNIVLIALSVLAVLKGLYNFATCGLVGLVTF
LLLCGRSCTTSLYKGVYELQTLELNMETLNMTMPLSCTKNNSHHYIMVG
NETGLELTLTNTSIINHKFCNLSDAHKKNLYDHALMSIISTFHLSIPNF
NQYEAMSCDFNGGKISVQYNLSHSYAGDAANHCGTVANGVLQTFMRMAW
GGSYIALDSGGCGNWDCIMTSYQYLIIQNTTWEDHCQFSRPSPIGYLGL
LSQRTRDIYISRRRRGTFTWTLSDSEGKDTPGGYCLTRWMLIEAELKCF
GNTAVAKCNEKHDEEFCDMLRLFDFNKQAIQRCKAEAQMSIQLINKAVN
ALINDQLIMKNHLRDIMGIPYCNYSKYWYLNHTTTGRTSLPKCWLVSNG
SYLNETHFSDDIEQQADNMITEMLQKEYMERQGKTPLGLVDLFVFSTSF
YLISIFLHLVKIPTHRHIVGKSCPKPHRLNHMGICSCGLYKQPGVPVKW
KR

### DETAILED DESCRIPTION

LASV is an enveloped RNA virus covered with glycoprotein complex (GPC) trimer spikes (Li et al., PLOS Pathog 12, e1005418, 2016; Schlie et al., J Virol 84, 983-992, 2010), which are the major viral antigen. Each protomer of the GPC trimer consists of a receptor-binding GP1 subunit, a transmembrane-spanning GP2 subunit, and the stable signal peptide (SSP), which originates from the precursor for proper processing and function (Hastie et al., Science 356, 923-928, 2017; Igonet et al., Proc Natl Acad Sci USA 108, 19967-19972, 2011; York and Nunberg, J Virol 90, 8341-8350, 2016; Eichler et al., EMBO Rep 4, 1084-1088, 2003). The majority of LASV vaccine development efforts have focused on the glycoprotein complex (GPC) present on the virion envelope. However, there has been very limited success due to the highly glycosylated GPC inducing only a weak and inconsistent immune response in both natural infection and vaccination (Baize et al., J Virol 83, 5890-5903, 2009; Fisher-Hoch et al., J Virol 74, 6777-6783, 2000; Sommerstein et al., PLOS Pathog 11, e1005276, 2015; Warner et al., Drug Des Devel Ther 12, 2519-2527, 2018; Lukashevich and Pushko, Expert Rev Vaccines 15, 1135-1150, 2016). The limited number of LASV neutralizing antibodies isolated further impedes LASV vaccine research, as neutralizing antibodies are critical to providing insights to vaccine design (Kwong et al., Cold Spring Harb Perspect Med 1, a007278, 2011). This disclosue provides LASV GPC ectodomain trimer stabilised in a prefusion conformation that are shown to elicit a neutralizing immune response to LASV in animal models. In some embodiments, the LASV GPC ectodomain trimer is linked to encapsulin nanoparticles using the spytag:spycatcher system to generate an immunogen for use to elicit a neutralizing immune response to LASV in a subject.

### I. Terms and Methods

Unless otherwise noted, technical terms are used according to conventional usage. Definitions of common terms in molecular biology may be found in Krebs et al. (eds.), Lewin's genes XII, published by Jones & Bartlett Learning, 2017; and Meyers et al. (eds.), The Encyclopedia of Cell Biology and Molecular Medicine, published by Wiley-VCH in 16 volumes, 2008; and other similar references.

As used herein, the singular forms “a,” “an,” and “the,” refer to both the singular as well as plural, unless the context clearly indicates otherwise. For example, the term “an antigen” includes single or plural antigens and can be considered equivalent to the phrase “at least one antigen.” As used herein, the term “comprises” means “includes.” It is further to be understood that any and all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particularly suitable methods and materials are described herein. In case of conflict, the present specification, including explanations of terms, will control. In addition, the materials, methods, and examples are illustrative only and not intended to be limiting. To facilitate review of the various embodiments, the following explanations of terms are provided:

Adjuvant: A component of an immunogenic composition used to enhance antigenicity. In some embodiments, an adjuvant can include a suspension of minerals (alum, aluminum hydroxide, or phosphate) on which antigen is adsorbed; or water-in-oil emulsion, for example, in which antigen solution is emulsified in mineral oil (Freund incomplete adjuvant), sometimes with the inclusion of killed mycobacteria (Freund's complete adjuvant) to further enhance antigenicity (inhibits degradation of antigen and/or causes influx of macrophages). In some embodiments, the adjuvant used in a disclosed immunogenic composition is a combination of lecithin and carbomer homopolymer (such as the ADJUPLEX™ adjuvant available from Advanced BioAdjuvants, LLC, see also Wegmann, Clin Vaccine Immunol, 22(9): 1004-1012, 2015). Additional adjuvants for use in the disclosed immunogenic compositions include the QS21 purified plant extract, Matrix M, AS01, MF59, and ALFQ adjuvants. Immunostimulatory oligonucleotides (such as those including a CpG motif) can also be used as adjuvants. Adjuvants include biological molecules (a “biological adjuvant”), such as costimulatory molecules. Exemplary adjuvants include IL-2, RANTES, GM-CSF, TNF-α, IFN-γ, G-CSF, LFA-3, CD72, B7-1, B7-2, OX-40L, 4-1BBL and toll-like receptor (TLR) agonists, such as TLR-9 agonists. See, also, Singh (ed.) Vaccine Adjuvants and Delivery Systems. Wiley-Interscience, 2007. Adjuvants can be used in combination with the disclosed immunogens.

Administration: The introduction of a composition into a subject by a chosen route. Administration can be local or systemic. For example, if the chosen route is intravenous, the composition (such as a composition including a disclosed immunogen) is administered by introducing the composition into a vein of the subject. Exemplary routes of administration include, but are not limited to, oral, injection (such as subcutaneous, intramuscular, intradermal, intraperitoneal, and intravenous), sublingual, rectal, transdermal (for example, topical), intranasal, vaginal, and inhalation routes.

Amino acid substitution: The replacement of one amino acid in a polypeptide with a different amino acid. In some examples, an amino acid in a polypeptide is substituted with an amino acid from a homologous polypeptide.

Antibody: An immunoglobulin, antigen-binding fragment, or derivative thereof, that specifically binds and recognizes an analyte (antigen), such as LASV GPC. The term “antibody” is used herein in the broadest sense and encompasses various antibody structures, including but not limited to monoclonal antibodies, polyclonal antibodies, multispecific antibodies (e.g., bispecific antibodies), and antibody fragments, so long as they exhibit the desired antigen-binding activity. Non-limiting examples of antibodies include, for example, intact immunoglobulins and variants and fragments thereof that retain binding affinity for the antigen. Examples of antibody fragments include but are not limited to Fv, Fab, Fab′, Fab′-SH, F(ab′)₂; diabodies; linear antibodies; single-chain antibody molecules (e.g. scFv); and multispecific antibodies formed from antibody fragments. Antibody fragments include antigen binding fragments either produced by the modification of whole antibodies or those synthesized de novo using recombinant DNA methodologies (see, e.g., Kontermann and Dubel (Ed), Antibody Engineering, Vols. 1-2, 2^{nd} Ed., Springer Press, 2010). Light and heavy chain variable regions contain a “framework” region interrupted by three hypervariable regions, also called “complementarity-determining regions” or “CDRs” (see, e.g., Kabat et al., Sequences of Proteins of Immunological Interest, U.S. Department of Health and Human Services, 1991). The framework region of an antibody, that is the combined framework regions of the constituent light and heavy chains, serves to position and align the CDRs in three-dimensional space. The CDRs are primarily responsible for binding to an epitope of an antigen.

Carrier: An immunogenic molecule to which an antigen (such as an LASV GPC ectodomain trimer) can be linked. When linked to a carrier, the antigen may become more immunogenic. Carriers are chosen to increase the immunogenicity of the antigen and/or to elicit antibodies against the carrier which are diagnostically, analytically, and/or therapeutically beneficial. Useful carriers include polymeric carriers, which can be natural (for example, proteins from bacteria or viruses), semi-synthetic or synthetic materials containing one or more functional groups to which a reactant moiety can be attached.

Conservative variants: “Conservative” amino acid substitutions are those substitutions that do not substantially affect or decrease a function of a protein, such as the ability of the protein to induce an immune response when administered to a subject. The term conservative variation also includes the use of a substituted amino acid in place of an unsubstituted parent amino acid. Furthermore, deletions or additions which alter, add or delete a single amino acid or a small percentage of amino acids (for instance less than 5%, in some embodiments less than 1%) in an encoded sequence are conservative variations where the alterations result in the substitution of an amino acid with a chemically similar amino acid.

The following six groups are examples of amino acids that are considered to be conservative substitutions for one another:
1) Alanine (A), Serine (S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).

Non-conservative substitutions are those that reduce an activity or function of the recombinant LASV GPC ectodomain trimer, such as the ability to induce an immune response when administered to a subject. For instance, if an amino acid residue is essential for a function of the protein, even an otherwise conservative substitution may disrupt that activity. Thus, a conservative substitution does not alter the basic function of a protein of interest.

Control: A reference standard. In some embodiments, the control is a negative control sample obtained from a healthy patient. In other embodiments, the control is a positive control sample obtained from a patient diagnosed with a LASV infection. In still other embodiments, the control is a historical control or standard reference value or range of values (such as a previously tested control sample, such as a group of patients infected with a LASV with known prognosis or outcome, or group of samples that represent baseline or normal values).

A difference between a test sample and a control can be an increase or conversely a decrease. The difference can be a qualitative difference or a quantitative difference, for example a statistically significant difference. In some examples, a difference is an increase or decrease, relative to a control, of at least about 5%, such as at least about 10%, at least about 20%, at least about 30%, at least about 40%, at least about 50%, at least about 60%, at least about 70%, at least about 80%, at least about 90%, at least about 100%, at least about 150%, at least about 200%, at least about 250%, at least about 300%, at least about 350%, at least about 400%, at least about 500%, or greater than 500%.

Covalent bond: An interatomic bond between two atoms, characterized by the sharing of one or more pairs of electrons by the atoms. The terms “covalently bound” or “covalently linked” refer to making two separate molecules into one contiguous molecule. The terms include reference to joining an antigen (such as an LASV GPC ectodomain trimer) either directly or indirectly to a carrier molecule, for example indirectly with an intervening linker molecule, such as a peptide or non-peptide linker.

Degenerate variant: In the context of the present disclosure, a “degenerate variant” refers to a polynucleotide encoding a polypeptide that includes a sequence that is degenerate as a result of the genetic code. There are 20 natural amino acids, most of which are specified by more than one codon. Therefore, all degenerate nucleotide sequences encoding a peptide are included as long as the amino acid sequence of the peptide encoded by the nucleotide sequence is unchanged.

Effective amount: An amount of agent, such as an immunogen, that is sufficient to elicit a desired response, such as an immune response in a subject. It is understood that to obtain a protective immune response against an antigen of interest can require multiple administrations of a disclosed immunogen, and/or administration of a disclosed immunogen as the “prime” in a prime boost protocol wherein the boost immunogen can be different from the prime immunogen. Accordingly, an effective amount of a disclosed immunogen can be the amount of the immunogen sufficient to elicit a priming immune response in a subject that can be subsequently boosted with the same or a different immunogen to elicit a protective immune response.

In one example, a desired response is to elicit an immune response that inhibits or prevents LASV infection. LASV infection does not need to be completely eliminated or prevented for the composition to be effective.

Epitope: An antigenic determinant. These are particular chemical groups or peptide sequences on a molecule that are antigenic, such that they elicit a specific immune response, for example, an epitope is the region of an antigen to which B and/or T cells respond. An antibody can bind to a particular antigenic epitope, such as an epitope on LASC GPC ectodomain. Epitopes can be formed both from contiguous amino acids or noncontiguous amino acids juxtaposed by tertiary folding of a protein.

Expression: Transcription or translation of a nucleic acid sequence. For example, a gene is expressed when its DNA is transcribed into an RNA or RNA fragment, which in some examples is processed to become mRNA. A gene may also be expressed when its mRNA is translated into an amino acid sequence, such as a protein or a protein fragment. In a particular example, a heterologous gene is expressed when it is transcribed into an RNA. In another example, a heterologous gene is expressed when its RNA is translated into an amino acid sequence. The term “expression” is used herein to denote either transcription or translation. Regulation of expression can include controls on transcription, translation, RNA transport and processing, degradation of intermediary molecules such as mRNA, or through activation, inactivation, compartmentalization or degradation of specific protein molecules after they are produced.

Expression Control Sequences: Nucleic acid sequences that regulate the expression of a heterologous nucleic acid sequence to which it is operatively linked. Expression control sequences are operatively linked to a nucleic acid sequence when the expression control sequences control and regulate the transcription and, as appropriate, translation of the nucleic acid sequence. Thus expression control sequences can include appropriate promoters, enhancers, transcription terminators, a start codon (ATG) in front of a protein-encoding gene, splicing signal for introns, maintenance of the correct reading frame of that gene to permit proper translation of mRNA, and stop codons. The term “control sequences” is intended to include, at a minimum, components whose presence can influence expression, and can also include additional components whose presence is advantageous, for example, leader sequences and fusion partner sequences. Expression control sequences can include a promoter.

A promoter is a minimal sequence sufficient to direct transcription. Also included are those promoter elements which are sufficient to render promoter-dependent gene expression controllable for cell-type specific, tissue-specific, or inducible by external signals or agents; such elements may be located in the 5′ or 3′ regions of the gene. Both constitutive and inducible promoters are included (see for example, Bitter et al., Methods in Enzymology 153:516-544, 1987). For example, when cloning in bacterial systems, inducible promoters such as pL of bacteriophage lambda, plac, ptrp, ptac (ptrp-lac hybrid promoter) and the like may be used. In one embodiment, when cloning in mammalian cell systems, promoters derived from the genome of mammalian cells (such as metallothionein promoter) or from mammalian viruses (such as the retrovirus long terminal repeat; the adenovirus late promoter; the vaccinia virus 7.5K promoter) can be used. Promoters produced by recombinant DNA or synthetic techniques may also be used to provide for transcription of the nucleic acid sequences.

Expression vector: A vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

Heterologous: A heterologous polypeptide or polynucleotide refers to a polypeptide or polynucleotide derived from a different source or species.

Host cells: Cells in which a vector can be propagated and its DNA expressed. The cell may be prokaryotic or eukaryotic. The term also includes any progeny of the subject host cell. It is understood that all progeny may not be identical to the parental cell since there may be mutations that occur during replication. However, such progeny are included when the term “host cell” is used.

Immune response: A response of a cell of the immune system, such as a B cell, T cell, or monocyte, to a stimulus. In one embodiment, the response is specific for a particular antigen (an “antigen-specific response”). In one embodiment, an immune response is a T cell response, such as a CD4+ response or a CD8+ response. In another embodiment, the response is a B cell response, and results in the production of specific antibodies.

Immunogenic conjugate: A composition composed of at least two heterologous molecules (such as an LASV GPC trimer and a carrier, such as a protein carrier) linked together that stimulates or elicits an immune response to a molecule in the conjugate in a vertebrate. In some embodiments where the conjugate include a viral antigen, the immune response is protective in that it enables the vertebrate animal to better resist infection from the virus from which the antigen is derived.

Immune response: A response of a cell of the immune system, such as a B cell, T cell, or monocyte, to a stimulus. In one embodiment, the response is specific for a particular antigen (an “antigen-specific response”). In one embodiment, an immune response is a T cell response, such as a CD4+ response or a CD8+ response. In another embodiment, the response is a B cell response, and results in the production of specific antibodies. “Priming an immune response” refers to treatment of a subject with a “prime” immunogen to induce an immune response that is subsequently “boosted” with a boost immunogen. Together, the prime and boost immunizations produce the desired immune response in the subject. “Enhancing an immune response” refers to co-administration of an adjuvant and an immunogenic agent, wherein the adjuvant increases the desired immune response to the immunogenic agent compared to administration of the immunogenic agent to the subject in the absence of the adjuvant.

Immunogen: A protein or a portion thereof that is capable of inducing an immune response in a mammal, such as a mammal infected or at risk of infection with a pathogen.

Immunogenic composition: A composition comprising a disclosed immunogen, or a nucleic acid molecule or vector encoding a disclosed immunogen, that elicits a measurable CTL response against the immunogen, or elicits a measurable B cell response (such as production of antibodies) against the immunogen, when administered to a subject. It further refers to isolated nucleic acids encoding an immunogen, such as a nucleic acid that can be used to express the immunogen (and thus be used to elicit an immune response against this immunogen). For in vivo use, the immunogenic composition will typically include the protein or nucleic acid molecule in a pharmaceutically acceptable carrier and may also include other agents, such as an adjuvant.

Inhibiting or treating a disease: Inhibiting the full development of a disease or condition, for example, in a subject who is at risk for a disease such as Lassa fever. “Treatment” refers to a therapeutic intervention that ameliorates a sign or symptom of a disease or pathological condition after it has begun to develop. The term “ameliorating,” with reference to a disease or pathological condition, refers to any observable beneficial effect of the treatment. Inhibiting a disease can include preventing or reducing the risk of the disease, such as preventing or reducing the risk of viral infection. The beneficial effect can be evidenced, for example, by a delayed onset of clinical symptoms of the disease in a susceptible subject, a reduction in severity of some or all clinical symptoms of the disease, a slower progression of the disease, a reduction in the viral load, an improvement in the overall health or well-being of the subject, or by other parameters that are specific to the particular disease. A “prophylactic” treatment is a treatment administered to a subject who does not exhibit signs of a disease or exhibits only early signs for the purpose of decreasing the risk of developing pathology.

Isolated: An “isolated” biological component has been substantially separated or purified away from other biological components, such as other biological components in which the component naturally occurs, such as other chromosomal and extrachromosomal DNA, RNA, and proteins. Proteins, peptides, nucleic acids, and viruses that have been “isolated” include those purified by standard purification methods. Isolated does not require absolute purity, and can include protein, peptide, nucleic acid, or virus molecules that are at least 50% isolated, such as at least 75%, 80%, 90%, 95%, 98%, 99%, or even 99.9% isolated.

Linked: The term “linked” means joined together, either directly or indirectly. For example, a first moiety may be covalently or noncovalently (e.g., electrostatically) linked to a second moiety. This includes, but is not limited to, covalently bonding one molecule to another molecule, noncovalently bonding one molecule to another (e.g. electrostatically bonding), non-covalently bonding one molecule to another molecule by hydrogen bonding, non-covalently bonding one molecule to another molecule by van der Waals forces, and any and all combinations of such couplings. Indirect attachment is possible, such as by using a “linker”. In several embodiments, linked components are associated in a chemical or physical manner so that the components are not freely dispersible from one another, at least until contacting a cell, such as an immune cell.

Lassa virus (LASV): A virus belonging to the genus Arenavirus, family Arenaviridae. Lassa virus has a segmented (two segments—small and large), single-stranded, ambisense RNA genome and a lipid envelope. The small genome segment encodes the nucleoprotein (NP) and the surface glycoprotein (GP) precursor. The large segment encodes a small zinc finger protein (Z) and an RNA polymerase (L). LASV is the causative agent of Lassa fever, which is endemic in Western Africa. Lassa fever is an acute viral hemorrhagic illness. Symptoms of Lassa fever include fever, weakness, cough, sore throat, headache and gastrointestinal illness.

LASV Glycoprotein complex (LASV GPC): An antigen expressed on the Lassa virus (LASV) surface. The form of GPC found on the LASV virion is a trimer of heterodimers, each containing GP1 (the receptor binding subunit) and GP2 (the transmembrane, fusion-mediating subunit). As the sole LASV surface antigen, GPC is the primary target of protective humoral immune responses (Hastie et al., Science 356, 923-928, 2017).

Linker: One or more molecules or groups of atoms positioned between two moieties. Typically, linkers are bifunctional, i.e., the linker includes a functional group at each end, wherein the functional groups are used to couple the linker to the two moieties. The two functional groups may be the same, i.e., a homobifunctional linker, or different, i.e., a heterobifunctional linker. In several embodiments, a peptide linker can be used to link the C-terminus of a first protein to the N-terminus of a second protein. Non-limiting examples of peptide linkers include glycine-serine peptide linkers, which are typically not more than 10 amino acids in length. Typically, such linkage is accomplished using molecular biology techniques to genetically manipulate DNA encoding the first polypeptide linked to the second polypeptide by the peptide linker.

Native protein, sequence, or disulfide bond: A polypeptide, sequence or disulfide bond that has not been modified, for example, by selective mutation. For example, selective mutation to focus the antigenicity of the antigen to a target epitope, or to introduce a disulfide bond into a protein that does not occur in the native protein. Native protein or native sequence are also referred to as wild-type protein or wild-type sequence. A non-native disulfide bond is a disulfide bond that is not present in a native protein, for example, a disulfide bond that forms in a protein due to introduction of one or more cysteine residues into the protein by genetic engineering.

Nucleic acid molecule: A polymeric form of nucleotides, which may include both sense and anti-sense strands of RNA, cDNA, genomic DNA, and synthetic forms and mixed polymers of the above. A nucleotide refers to a ribonucleotide, deoxynucleotide or a modified form of either type of nucleotide. The term “nucleic acid molecule” as used herein is synonymous with “nucleic acid” and “polynucleotide.” A nucleic acid molecule is usually at least 10 bases in length, unless otherwise specified. The term includes single- and double-stranded forms of DNA. A polynucleotide may include either or both naturally occurring and modified nucleotides linked together by naturally occurring and/or non-naturally occurring nucleotide linkages. “cDNA” refers to a DNA that is complementary or identical to an mRNA, in either single stranded or double stranded form. “Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked nucleic acid sequences are contiguous and, where necessary to join two protein-coding regions, in the same reading frame.

Pharmaceutically acceptable carriers: The pharmaceutically acceptable carriers of use are conventional. Remington: The Science and Practice of Pharmacy, 22^{nd} ed., London, UK: Pharmaceutical Press, 2013, describes compositions and formulations suitable for pharmaceutical delivery of the disclosed agents.

In general, the nature of the carrier will depend on the particular mode of administration being employed. For instance, parenteral formulations usually include injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, added preservatives (such as non-natural preservatives), and pH buffering agents and the like, for example sodium acetate or sorbitan monolaurate. In particular examples, the pharmaceutically acceptable carrier is sterile and suitable for parenteral administration to a subject for example, by injection. In some embodiments, the active agent and pharmaceutically acceptable carrier are provided in a unit dosage form such as a pill or in a selected quantity in a vial. Unit dosage forms can include one dosage or multiple dosages (for example, in a vial from which metered dosages of the agents can selectively be dispensed).

Polypeptide: Any chain of amino acids, regardless of length or post-translational modification (e.g., glycosylation or phosphorylation). “Polypeptide” applies to amino acid polymers including naturally occurring amino acid polymers and non-naturally occurring amino acid polymer as well as in which one or more amino acid residue is a non-natural amino acid, for example, an artificial chemical mimetic of a corresponding naturally occurring amino acid. A “residue” refers to an amino acid or amino acid mimetic incorporated in a polypeptide by an amide bond or amide bond mimetic. A polypeptide has an amino terminal (N-terminal) end and a carboxy terminal (C-terminal) end. “Polypeptide” is used interchangeably with peptide or protein, and is used herein to refer to a polymer of amino acid residues.

Prime-boost immunization: An immunotherapy including administration of multiple immunogens over a period of time to elicit the desired immune response.

Nanoparticle: a nanoscale moiety of from 1-1000 nm in diameter.

In some embodiments, a protein nanoparticle is provided that comprises a multi-subunit, self-assembling, protein-based polyhedron shaped structure optionally linked to a prefusion LASV GPC ectodomain trimer as described herein. The subunits are each composed of proteins, for example a glycosylated polypeptide. In some embodiments, protomers of the disclosed GPC ectodomain trimer can be fused to the subunits of the protein nanoparticles to provide multiple copies of the trimer on each protein nanoparticle. Non-limiting examples of protein nanoparticles include ferritin nanoparticles (see, e.g., Zhang, Y. Int. J. Mol. Sci., 12:5406-5421, 2011), encapsulin nanoparticles (see, e.g., Sutter et al., Nature Struct. and Mol. Biol., 15:939-947, 2008), Sulfur Oxygenase Reductase (SOR) nanoparticles (see, e.g., Urich et al., Science, 311:996-1000, 2006), lumazine synthase nanoparticles (see, e.g., Zhang et al., J. Mol. Biol., 306: 1099-1114, 2001), and pyruvate dehydrogenase nanoparticles (see, e.g., Izard et al., PNAS 96: 1240-1245, 1999). Ferritin, encapsulin, SOR, lumazine synthase, and pyruvate dehydrogenase are monomeric proteins that self-assemble into a globular protein complexes that in some cases consists of 24, 60, 24, 60, and 60 protein subunits, respectively. Additional protein nanoparticle structures are described by Heinze et al., J Phys Chem B., 120(26):5945-52, 2016; Hsia et al., Nature, 535(7610):136-9, 2016; and King et al., Nature, 510(7503): 103-8, 2014.

Recombinant: A recombinant nucleic acid is one that has a sequence that is not naturally occurring or has a sequence that is made by an artificial combination of two otherwise separated segments of sequence. This artificial combination can be accomplished, for example, the artificial manipulation of isolated segments of nucleic acids, for example, using genetic engineering techniques. A recombinant protein is one that has a sequence that is not naturally occurring or has a sequence that is made by an artificial combination of two otherwise separated segments of sequence. In several embodiments, a recombinant protein is encoded by a heterologous (for example, recombinant) nucleic acid that has been introduced into a host cell, such as a bacterial or eukaryotic cell. The nucleic acid can be introduced, for example, on an expression vector having signals capable of expressing the protein encoded by the introduced nucleic acid or the nucleic acid can be integrated into the host cell chromosome.

Sequence identity: The similarity between amino acid sequences is expressed in terms of the similarity between the sequences, otherwise referred to as sequence identity. Sequence identity is frequently measured in terms of percentage identity; the higher the percentage, the more similar the two sequences are. Homologs, orthologs, or variants of a polypeptide will possess a relatively high degree of sequence identity when aligned using standard methods.

Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. In the Biosciences 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations.

Variants of a polypeptide are typically characterized by possession of at least about 75%, for example, at least about 80%, 85%, 90%, 91%, 92%, 93%, 94%, 95%, 96%, 97%, 98% or 99% sequence identity counted over the full length alignment with the amino acid sequence of interest. Proteins with even greater similarity to the reference sequences will show increasing percentage identities when assessed by this method, such as at least 80%, at least 85%, at least 90%, at least 95%, at least 98%, or at least 99% sequence identity. When less than the entire sequence is being compared for sequence identity, homologs and variants will typically possess at least 80% sequence identity over short windows of 10-20 amino acids, and may possess sequence identities of at least 85% or at least 90% or 95% depending on their similarity to the reference sequence. Methods for determining sequence identity over such short windows are available at the NCBI website on the internet.

As used herein, reference to “at least 90% identity” (or similar language) refers to “at least 90%, at least 91%, at least 92%, at least 93%, at least 94%, at least 95%, at least 96%, at least 97%, at least 98%, at least 99%, or even 100% identity” to a specified reference sequence.

Signal Peptide: A short amino acid sequence (e.g., approximately 10-35 amino acids in length) that directs newly synthesized secretory or membrane proteins to and through membranes (for example, the endoplasmic reticulum membrane). Signal peptides are typically located at the N-terminus of a polypeptide and are removed by signal peptidases. Signal peptide sequences typically contain three common structural features: an N-terminal polar basic region (n-region), a hydrophobic core, and a hydrophilic c-region).

Specifically bind: When referring to the formation of an antibody:antigen protein complex, or a protein:protein complex, refers to a binding reaction which determines the presence of a target protein, peptide, or polysaccharide (for example, a glycoprotein), in the presence of a heterogeneous population of proteins and other biologics. Thus, under designated conditions, a particular antibody or protein binds preferentially to a particular target protein, peptide or polysaccharide (such as an antigen present on the surface of a pathogen, for example, gp120) and does not bind in a significant amount to other proteins or polysaccharides present in the sample or subject. Specific binding can be determined by standard methods. A first protein or antibody specifically binds to a target protein when the interaction has a K_{D} of less than 10⁻⁷ Molar, such as less than 10⁻⁸ Molar, less than 10⁻⁹, or even less than 10⁻¹⁰ Molar.

Soluble protein: A protein capable of dissolving in aqueous liquid at room temperature and remaining dissolved. The solubility of a protein may change depending on the concentration of the protein in the water-based liquid, the buffering condition of the liquid, the concentration of other solutes in the liquid, for example salt and protein concentrations, and the heat of the liquid. In several embodiments, a soluble protein is one that dissolves to a concentration of at least 0.5 mg/ml in phosphate buffered saline (pH 7.4) at room temperature and remains dissolved for at least 48 hours.

Subject: Living multicellular vertebrate organisms, a category that includes human and non-human mammals. In an example, a subject is a human. In an additional example, a subject is selected that is in need of inhibiting of an LASV infection. For example, the subject is either uninfected and at risk of LASV infection or is infected in need of treatment.

T4 Fibritin trimerization domain: Also referred to as a “foldon” domain, the T4 Fibritin trimerization domain comprises an amino acid sequence that naturally forms a trimeric structure. In some examples, a T4 Fibritin trimerization domain can be linked to the C-terminus of a disclosed recombinant LASV GPC ectodomain. In one example, a T4 Fibritin trimerization domain comprises the amino acid sequence set forth as (GYIPEAPRDGQAYVRKDGEWVLLSTF (SEQ ID NO: 4). In some embodiments, a protease cleavage site (such as a thrombin cleavage site) can be included between the C-terminus of the recombinant LASV GPC ectodomain and the T4 Fibritin trimerization domain to facilitate removal of the trimerization domain as needed, for example, following expression and purification of the recombinant LASV GPC ectodomain.

Transmembrane domain: An amino acid sequence that inserts into a lipid bilayer, such as the lipid bilayer of a cell or virus or virus-like particle. A transmembrane domain can be used to anchor an antigen to a membrane.

Under conditions sufficient for: A phrase that is used to describe any environment that permits a desired activity.

Vaccine: A pharmaceutical composition that elicits a prophylactic or therapeutic immune response in a subject. In some cases, the immune response is a protective immune response. Typically, a vaccine elicits an antigen-specific immune response to an antigen of a pathogen, for example a viral pathogen, or to a cellular constituent correlated with a pathological condition. A vaccine may include a polynucleotide (such as a nucleic acid encoding a disclosed antigen), a peptide or polypeptide (such as a disclosed antigen), a virus, a cell or one or more cellular constituents. In one specific, non-limiting example, a vaccine reduces the severity of the symptoms associated with LASV infection and/or decreases the viral load compared to a control. In another non-limiting example, a vaccine reduces LASV infection compared to a control.

Vector: An entity containing a DNA or RNA molecule bearing a promoter(s) that is operationally linked to the coding sequence of an immunogenic protein of interest and can express the coding sequence. Non-limiting examples include a naked or packaged (lipid and/or protein) DNA, a naked or packaged RNA, a subcomponent of a virus or bacterium or other microorganism that may be replication-incompetent, or a virus or bacterium or other microorganism that may be replication-competent. A vector is sometimes referred to as a construct. Recombinant DNA vectors are vectors having recombinant DNA. A vector can include nucleic acid sequences that permit it to replicate in a host cell, such as an origin of replication. A vector can also include one or more selectable marker genes and other genetic elements. Viral vectors are recombinant nucleic acid vectors having at least some nucleic acid sequences derived from one or more viruses.

A non-limiting example of a DNA-based expression vector is pCDNA3.1, which can include includes a mammalian expression enhancer and promoter (such as a CMV promoter). Non-limiting examples of viral vectors include adeno-associated virus (AAV) vectors as well as Poxvirus vector (e.g., Vaccinia, MVA, avian Pox, or Adenovirus).

Virus-like particle (VLP): A non-replicating, viral shell, derived from any of several viruses. VLPs are generally composed of one or more viral proteins, such as, but not limited to, those proteins referred to as capsid, coat, shell, surface and/or envelope proteins, or particle-forming polypeptides derived from these proteins. VLPs can form spontaneously upon recombinant expression of the protein in an appropriate expression system. The presence of VLPs following recombinant expression of viral proteins can be detected using conventional techniques, such as by electron microscopy, biophysical characterization, and the like. Further, VLPs can be isolated by known techniques, e.g., density gradient centrifugation and identified by characteristic density banding. See, for example, Baker et al. (1991) Biophys. J. 60:1445-1456; and Hagensee et al. (1994) J. Virol. 68:4503-4505; Vincente, J Invertebr Pathol., 2011; Schneider-Ohrum and Ross, Curr. Top. Microbiol. Immunol., 354: 53073, 2012).

### II. Recombinant LASV GPC

Disclosed herein are recombinant LASV GPC ectodomain trimers comprising protomers comprising one or more amino acid substitutions that inhibit a conformational change in LASV GPC from the prefusion conformation to the postfusion conformation, and therefore stabilize the LASV GPC ectodomain trimer in the prefusion conformation. The recombinant LASV GPC ectodomain trimer produces a superior immune response compared to corresponding LASV GPC ectodomain trimer that is not stabilized in the prefusion conformation.

An exemplary sequence of native LASV GPC protein (including the signal peptide, ectodomain and TM and CT domains) is provided as SEQ ID NO: 1:

MGQIVTFFQEVPHVIEEVMNIVLIALSVLAVLKGLYNFATCGLVGLVTF
LLLCGRSCTTSLYKGVYELQTLELNMETLNMTMPLSCTKNNSHHYIMVG
NETGLELTLTNTSIINHKFCNLSDAHKKNLYDHALMSIISTFHLSIPNF
NQYEAMSCDFNGGKISVQYNLSHSYAGDAANHCGTVANGVLQTFMRMAW
GGSYIALDSGRGNWDCIMTSYQYLIIQNTTWEDHCQFSRPSPIGYLGLL
SQRTRDIYISRRLLGTFTWTLSDSEGKDTPGGYCLTRWMLIEAELKCFG
NTAVAKCNEKHDEEFCDMLRLFDFNKQAIQRLKAEAQMSIQLINKAVNA
LINDQLIMKNHLRDIMGIPYCNYSKYWYLNHTTTGRTSLPKCWLVSNGS
YLNETHFSDDIEQQADNMITEMLQKEYMERQGKTPLGLVDLFVFSTSFY
LISIFLHLVKIPTHRHIVGKSCPKPHRLNHMGICSCGLYKQPGVPVKWK
R

The amino acid numbering used herein for residues of the LASV GPC is with reference to the LASV GPC sequence provided as SEQ ID NO: 1. With reference to the LASV GPC sequence provided as SEQ ID NO: 1, the ectodomain of the LASV GPC includes about residues 59-418. Residues 1-58 are the signal peptide, which is removed during cellular processing. The GP1/GP2 cleavage site is located at position 259/260. The C-terminal end of the GP2 ectodomain is located at about residue 418. In some embodiments, the protomers of the prefusion-stabilized LASV GPC ectodomain trimer can have a C-terminal residue (which can be linked to a trimerization domain, or a transmembrane domain, for example) of the C-terminal residue of the ectodomain (e.g., position 418), or from one of positions 410-418. The position numbering of the GPC protein may vary between LASV strains, but the sequences can be aligned to determine relevant structural domains and cleavage sites. It will be appreciated that a few residues (such as up to 10) on the N- and C-terminal ends of the ectodomain can be removed or modified in the disclosed immunogens without decreasing the utility of the GPC ectodomain trimer as an immunogen.

In some embodiments, the immunogen comprises a recombinant LASV GPC ectodomain trimer stabilized in a prefusion conformation by one or more amino acid substitutions in protomers of the trimer, the amino acid substitutions comprising cysteine substitutions at GPC positions 207 and 326 (such as R207C and L326C substitutions) that form a non-natural interprotomer disulfide bond. In some such embodiments, the protomers further comprise a glycine insertion between GPC positions 205 and 206. For example, the protomers comprise R207GC and L326C substitutions that form a non-natural inter-protomer disulfide bond.

In some embodiments, the protomers of the GPC ectodomain comprise a GP1 protein comprising or consisting of GPC positions 59-259 and a GP2 ectodomain comprising or consisting of GPC positions 260-418.

In some embodiments, the protomers of the GPC ectodomain further comprise amino acid substitutions to add arginine residues to a GP1/GP2 cleavage site at positions 256-259. For example, the protomers of the trimer comprise L258R and L259R substitutions.

An exemplary sequence of LASV GPC ectodomain including a R207GC and L326C substitutions substitution for stabilization in the prefusion conformation and L258R and L259R substitutions for enhancement of the GP1/GP2 cleavage site is provided as residues 59-418 of SEQ ID NO: 2:

TSLYKGVYELQTLELNMETLNMTMPLSCTKNNSHHYIMVGNETGLELTL
TNTSIINHKFCNLSDAHKKNLYDHALMSIISTFHLSIPNFNQYEAMSCD
FNGGKISVQYNLSHSYAGDAANHCGTVANGVLQTFMRMAWGGSYIALDS
GGCGNWDCIMTSYQYLIIQNTTWEDHCQFSRPSPIGYLGLLSQRTRDIY
ISRRRRGTFTWTLSDSEGKDTPGGYCLTRWMLIEAELKCFGNTAVAKCN
EKHDEEFCDMLRLFDFNKQAIQRCKAPAQMSIQLINKAVNALINDQLIM
KNHLRDIMGIPYCNYSKYWYLNHTTTGRTSLPKCWLVSNGSYLNETHFS
DDIEQQADNMITEMLQKE

In some embodiments, the recombinant LASV GPC ectodomain trimer comprises protomers comprising a sequence at least 90% (such as at least 95%, at least 98%, or at least 99%) identical to the ectodomain sequence of SEQ ID NO: 2, wherein the LASV GPC ectodomain trimer is stabilized in the prefusion conformation with one or more of the modifications provided herein (such as the R207GC and L326C substitutions and optionally L258R and L259R substitutions). In some embodiments, the recombinant LASV GPC ectodomain trimer comprises protomers comprising a sequence at least 90% (such as at least 95%, at least 98%, or at least 99%) identical to residues 59-418 of SEQ ID NO: 2, wherein the LASV GPC ectodomain trimer is stabilized in the prefusion conformation with one or more of the modifications provided herein (such as the R207GC and L326C substitutions and optionally L258R and L259R substitutions). In some embodiments, the recombinant LASV GPC ectodomain trimer comprises protomers comprising a sequence set forth as residues 59-418 of SEQ ID NO: 2

In alternative embodiments, the protomers of the trimer further comprise a mutation to remove a GP1/GP2 furin cleavage site of the GPC ectodomain, such as a GP1/GP2 furin cleavage site at positions 256-259. In some such embodiments, the mutation to remove the GP1/GP2 furin cleavage site comprises a deletion of GPC positions 256-259 with positions 255 and 260 fused by a peptide linker (such as a glycine or glycine-serine linker, for example a Gly-Gly-Gly linker.

In some embodiments, the C-terminal residue of the ectodomains of the protomers in the recombinant LASV GPC ectodomain trimer can be linked to a trimerization domain to promote trimerization of the protomers, and to stabilize the membrane proximal aspect of the protomers in a trimeric configuration. Non-limiting examples of exogenous multimerization domains that promote stable trimers of soluble recombinant proteins include: the GCN4 leucine zipper (Harbury et al. 1993 Science 262:1401-1407), the trimerization motif from the lung surfactant protein (Hoppe et al. 1994 FEBS Lett 344:191-195), collagen (McAlinden et al. 2003 J Biol Chem 278:42200-42207), and the phage T4 fibritin (Miroshnikov et al. 1998 Protein Eng 11:329-414), any of which can be linked to a recombinant LASV GPC ectodomain described herein (e.g., by linkage to the C-terminus of GP2 ectodomain) to promote trimerization of the recombinant LASV GPC ectodomain.

In some examples, the C-terminal residue of the GP2 ectodomain can be linked to a T4 fibritin domain. In specific examples, the T4 fibritin domain can include the amino acid sequence GYIPEAPRDGQAYVRKDGEWVLLSTF (SEQ ID NO: 4), which adopts a β-propeller conformation, and can fold and trimerize in an autonomous way (Tao et al. 1997 Structure 5:789-798).

Optionally, the heterologous trimerization is connected to the recombinant LASV GPC ectodomain via a peptide linker, such as an amino acid linker. Non-limiting examples of peptide linkers that can be used include glycine, serine, and glycine-serine linkers.

An exemplary sequence of LASV GPC ectodomain including R207GC and L326C substitutions that form a disulfide bond for stabilization in the prefusion conformation, L258R and L259R substitutions for enhancement of the GP1/GP2 cleavage site, and linked to a T4 fibritin trimerization domain is provided as residues 59-448 of SEQ ID NO: 2:

TSLYKGVYELQTLELNMETLNMTMPLSCTKNNSHHYIMVGNETGLELTL
TNTSIINHKFCNLSDAHKKNLYDHALMSIISTFHLSIPNFNQYEAMSCD
FNGGKISVQYNLSHSYAGDAANHCGTVANGVLQTFMRMAWGGSYIALDS
GGCGNWDCIMTSYQYLIIQNTTWEDHCQFSRPSPIGYLGLLSQRTRDIY
ISRRRRGTFTWTLSDSEGKDTPGGYCLTRWMLIEAELKCFGNTAVAKCN
EKHDEEFCDMLRLFDFNKQAIQRCKAPAQMSIQLINKAVNALINDQLIM
KNHLRDIMGIPYCNYSKYWYLNHTTTGRTSLPKCWLVSNGSYLNETHFS
DDIEQQADNMITEMLQKEGGGYIPEAPRDGQAYVRKDGEWVLLSTFL

In some embodiments, the recombinant LASV GPC ectodomain trimer comprises protomers comprising a sequence at least 90% (such as at least 95%, at least 98%, or at least 99%) identical to the ectodomain sequence of SEQ ID NO: 2 that are each linked to a trimerization domain, such as a T4 Fibritin trimerization domain, wherein the LASV GPC ectodomain trimer is stabilized in the prefusion conformation with one or more of the modifications provided herein (such as the R207GC and L326C substitutions and optionally L258R and L259R substitutions). In some embodiments, the recombinant LASV GPC ectodomain trimer comprises protomers linked to a trimerization domain, such as a T4 Fibritin trimerization domain, and comprising a sequence at least 90% (such as at least 95%, at least 98%, or at least 99%) identical to residues 59-448 of SEQ ID NO: 2, wherein the LASV GPC S ectodomain trimer is stabilized in the prefusion conformation with one or more of the modifications provided herein (such as the R207GC and L326C substitutions and optionally L258R and L259R substitutions). In some embodiments, the recombinant LASV GPC ectodomain trimer comprises protomers linked to a T4 Fibritin trimerization domain and comprising a sequence set forth as residues 59-448 of SEQ ID NO: 2.

In some embodiments, the LASV GPC ectodomain trimer (with or without trimerization domain) is linked to a heterologous moiety, such as a heterologous protein. In some embodiments, the protomers of the trimer linked to trimerization domain are fused C-terminally to a linkage tag or a purification tag, such as a spytag moiety. For example, the protomers of the trimer linked to trimerization domain are fused C-terminally to a spytag moiety that allows subsequent linkage to a heterologous moiety (such as a self-assembling protein nanoparticle) containing a corresponding spycatcher moiety. Optionally, a linker (such as a glycine, serine, or glycine-serine linker) can be included between the trimerization domain and the linkage tag or purification tag.

An exemplary sequence of LASV GPC ectodomain including R207GC and L326C substitutions that form a disulfide bond for stabilization in the prefusion conformation, L258R and L259R substitutions for enhancement of the GP1/GP2 cleavage site, and linked to a T4 fibritin trimerization domain and a spytag moiety is provided as residues 59-468 of SEQ ID NO: 2:

TSLYKGVYELQTLELNMETLNMTMPLSCTKNNSHHYIMVGNETGLELTL
TNTSIINHKFCNLSDAHKKNLYDHALMSIISTFHLSIPNFNQYEAMSCD
FNGGKISVQYNLSHSYAGDAANHCGTVANGVLQTFMRMAWGGSYIALDS
GGCGNWDCIMTSYQYLIIQNTTWEDHCQFSRPSPIGYLGLLSQRTRDIY
ISRRRRGTFTWTLSDSEGKDTPGGYCLTRWMLIEAELKCFGNTAVAKCN
EKHDEEFCDMLRLFDFNKQAIQRCKAPAQMSIQLINKAVNALINDQLIM
KNHLRDIMGIPYCNYSKYWYLNHTTTGRTSLPKCWLVSNGSYLNETHFS
DDIEQQADNMITEMLQKEGGGYIPEAPRDGQAYVRKDGEWVLLSTFLGG
GSGAHIVMVDAYKPTKGG

In some embodiments, the recombinant LASV GPC ectodomain trimer comprises protomers comprising a sequence at least 90% (such as at least 95%, at least 98%, or at least 99%) identical to the ectodomain sequence of SEQ ID NO: 2 that are each linked to a trimerization domain (such as a T4 Fibritin trimerization domain) and a spytag moiety, wherein the LASV GPC ectodomain trimer is stabilized in the prefusion conformation with one or more of the modifications provided herein (such as the R207GC and L326C substitutions and optionally L258R and L259R substitutions). In some embodiments, the recombinant LASV GPC ectodomain trimer comprises protomers linked to a trimerization domain, such as a T4 Fibritin trimerization domain, and a spytag, and comprising a sequence at least 90% (such as at least 95%, at least 98%, or at least 99%) identical to residues 59-468 of SEQ ID NO: 2, wherein the LASV GPC ectodomain trimer is stabilized in the prefusion conformation with one or more of the modifications provided herein (such as the R207GC and L326C substitutions and optionally L258R and L259R substitutions). In some embodiments, the recombinant LASV GPC ectodomain trimer comprises protomers linked to a T4 Fibritin trimerization domain and spytag and comprising a sequence set forth as residues 59-468 of SEQ ID NO: 2.

In some embodiments, the LASV GPC ectodomain trimer can be membrane anchored, for example, for embodiments where the LASV GPC ectodomain trimer is expressed as an attenuated viral vaccine, or a virus like particle, or by recombinant nucleic acid (such as mRNA). In such embodiments, the protomers in the trimer typically each comprise a C-terminal linkage to a transmembrane domain, such as the transmembrane domain (and optionally the cytosolic tail) of LASV GPC. In some embodiments, one or more peptide linkers (such as a gly-ser linker, for example, a 10 amino acid glycine-serine peptide linker can be used to link the recombinant LASV GPC ectodomain protomer to the transmembrane domain. The protomers linked to the transmembrane domain can include any of the stabilizing mutations provided herein (or combinations thereof) as long as the recombinant LASV GPC ectodomain trimer linked to the transmembrane domain retains the desired properties (e.g., the LASV GPC prefusion conformation).

An exemplary sequence of full length LASV GPC including R207GC and L326C substitutions that form a disulfide bond for stabilization in the prefusion conformation, and L258R and L259R substitutions for enhancement of the GP1/GP2 cleavage site, is provided as SEQ ID NO: 3:

MGQIVTFFQEVPHVIEEVMNIVLIALSVLAVLKGLYNFATCGLVGLVTF
LLLCGRSCTTSLYKGVYELQTLELNMETLNMTMPLSCTKNNSHHYIMVG
NETGLELTLTNTSIINHKFCNLSDAHKKNLYDHALMSIISTFHLSIPNF
NQYEAMSCDFNGGKISVQYNLSHSYAGDAANHCGTVANGVLQTFMRMAW
GGSYIALDSGGCGNWDCIMTSYQYLIIQNTTWEDHCQFSRPSPIGYLGL
LSQRTRDIYISRRRRGTFTWTLSDSEGKDTPGGYCLTRWMLIEAELKCF
GNTAVAKCNEKHDEEFCDMLRLFDFNKQAIQRCKAEAQMSIQLINKAVN
ALINDQLIMKNHLRDIMGIPYCNYSKYWYLNHTTTGRTSLPKCWLVSNG
SYLNETHFSDDIEQQADNMITEMLQKEYMERQGKTPLGLVDLFVFSTSF
YLISIFLHLVKIPTHRHIVGKSCPKPHRLNHMGICSCGLYKQPGVPVKW
KR

In some embodiments, the recombinant LASV GPC ectodomain trimer comprises protomers comprising the ectodomain sequence of SEQ ID NO: 3 that are each linked to a transmembrane domain and or a cytoplasmic tail. In some embodiments, the recombinant LASV GPC ectodomain trimer comprises protomers linked to a transmembrane domain comprising residues 59-492 of SEQ ID NO: 3. In some embodiments, the recombinant LASV GPC ectodomain trimer comprises protomers linked to a transmembrane domain comprising a sequence at least 90% (such as at least 95%, at least 98%, or at least 99%) identical to residues 59-492 of SEQ ID NO: 3, wherein the LASV GPC ectodomain trimer is stabilized in the prefusion conformation with one or more of the modifications provided herein (such as the such as the R207GC and L326C substitutions and optionally L258R and L259R substitutions).

The recombinant LASV GPC ectodomain trimer and variants thereof can be produced using recombinant techniques, or chemically or enzymatically synthesized.

Analogs and variants of the recombinant LASV GPC ectodomain trimer may be used in the methods and systems of the present disclosure. Through the use of recombinant DNA technology, variants of the recombinant LASV GPC ectodomain trimer may be prepared by altering the underlying DNA. All such variations or alterations in the structure of the recombinant LASV GPC ectodomain trimer resulting in variants are included within the scope of this disclosure. Such variants include insertions, substitutions, or deletions of one or more amino acid residues, glycosylation variants, unglycosylated recombinant LASV GPC ectodomain trimer, organic and inorganic salts, covalently modified derivatives of the recombinant LASV GPC ectodomain trimer, or a precursor thereof. Such variants may maintain one or more of the functional, biological activities of the recombinant LASV GPC ectodomain trimer, such as binding to cell surface receptor. The recombinant LASV GPC ectodomain trimer thereof can be modified, for example, by PEGylation, to increase the half-life of the protein in the recipient, and/or to make the protein more stable for delivery to a subject.

In some embodiments, a recombinant LASV GPC ectodomain trimer useful within the disclosure is modified by replacement of one or more naturally occurring side chains of the 20 genetically encoded amino acids (or D-amino acids) with other side chains, for example with groups such as alkyl, lower alkyl, cyclic 4-, 5-, 6-, to 7-membered alkyl, amide, amide lower alkyl, amide di(lower alkyl), lower alkoxy, hydroxy, carboxy and the lower ester derivatives thereof, and with 4-, 5-, 6-, to 7-membered heterocyclics. For example, proline analogs can be made in which the ring size of the proline residue is changed from a 5-membered ring to a 4-, 6-, or 7-membered ring. Cyclic groups can be saturated or unsaturated, and if unsaturated, can be aromatic or non-aromatic. Heterocyclic groups can contain one or more nitrogen, oxygen, and/or sulphur heteroatoms.

### Nanoparticles Containing LASV GPC Ectodomain Trimers

In some embodiments a protein nanoparticle (such as a self-assembling protein nanoparticle) is provided that includes a recombinant LASV GPC ectodomain trimer displayed on its surface. Any suitable nanoparticle format can be used.

In some embodiments, to construct protein nanoparticles, nucleic acid encoding a protomer of the LASV GPC ectodomain trimer can be fused to nucleic acid encoding a subunit of the protein nanoparticle (such as a ferritin protein, an encapsulin protein, a SOR protein, or a lumazine synthase protein) and expressed in cells under appropriate conditions. The fusion protein self-assembles into a nanoparticle any can be purified.

In several embodiments, to construct such protein nanoparticles, a purified LASV GPC ectodomain trimer can be linked (for example, via bioconjugation) to subunits of a purified self-assembling protein nanoparticle (such as a ferritin protein, an encapsulin protein, a SOR protein, or a lumazine synthase protein) and the resulting nanoparticle/S trimer purified.

In some embodiments, the LASV GPC ectodomain trimer is included in a self-assembling protein nanocage that directs its own release from cells inside small vesicles in a manner that resembles viruses, for example, as described in Votteler et al., “Designed proteins induce the formation of nanocage-containing extracellular vesicles,” Nature 540, 292-29, 2016. This hybrid biomaterial can fuse its membranes with target cells and deliver its contents, thereby transferring cargoes from one cell to another.

In some embodiments, the LASV GPC ectodomain trimer is linked to a self-assembling protein nanoparticle. Non-limiting example of self-assembling protein nanoparticles include ferritin nanoparticles, encapsulin nanoparticles, Sulfur Oxygenase Reductase (SOR) nanoparticles, and lumazine synthase nanoparticles, which are comprised of an assembly of monomeric subunits including ferritin proteins, encapsulin proteins, SOR proteins, and lumazine synthase, respectively. Additional protein nanoparticle structures are described by Heinze et al., J Phys Chem B., 120(26):5945-52, 2016; Hsia et al., Nature, 535(7610):136-9, 2016; and King et al., Nature, 510(7503): 103-8, 2014.

Linkage of the LASV GPC ectodomain trimer to the nanoparticle surface may be accomplished using any suitable means. In some embodiments, the protomers of the LASV GPC ectodomain trimer are C-terminally fused to subunits of the self-assembling nanoparticle, for example, as described in Kanekiyo et al. (Nature, 499:102-106, 2013) for ferritin-based nanoparticles and Sutter et al. (Nature Struct. and Mol. Biol., 15:939-947, 2008) for encapsulin-based nanoparticles).

In some embodiments, the LASV GPC ectodomain trimer is linked to the protein nanoparticle using the isopeptide bond conjugation system referred to as the SpyTag:SpyCatcher system (Brune, K. D. et al. Plug-and-Display: decoration of Virus-Like Particles via isopeptide bonds for modular immunization. Sci Rep 6, 19234, 2016) to display antigens on nanoparticle surface. The SpyTag:SpyCatcher system is highly specific and stable with an isopeptide bond and has been used for conjugation of antigens on nanoparticle surfaces (See, e.g., WO2011098772, U.S. Pat. Nos. 9,547,003, 10,247,727, and 10,527,609, Zakeri, B. et al. “Peptide tag forming a rapid covalent bond to a protein, through engineering a bacterial adhesin.” Proc Natl Acad Sci USA 109, E690-697, (2012); Brune, K. D. et al. Plug-and-Display: decoration of Virus-Like Particles via isopeptide bonds for modular immunization. Sci Rep 6, 19234, 2016). In such embodiments, the LASV GPC ectodomain trimer and the self-assembling protein nanoparticles are separately produced with fusion to either the spytag or spycatcher moiety and then combined under conditions suitable for isopeptide bond formation between the spytag and sypcatcher moieties.

In some embodiments, the LASV GPC ectodomain trimer is linked to the protein nanoparticle using the SpyTag:SpyCatcher system, wherein the protomers of the LASV GPC ectodomain trimer are C-terminally fused to spytag (as in SEQ ID NO: 2) and the protein nanoparticle is a self-assembled encapsulin nanoparticle with subunits fused to the spycatcher moiety. A non-limiting example of an encapsulin subunit sequence fused to spycatcher for use in the embodiments provided herein is set forth as SEQ ID NO: 5:

PYGWEYAAHPLCEVEVLSDENEVVKWGLRKSLPLIELRATFTLLWELDN
LECGKPNVDLSSLEETVRKVAEFEDEVIFRGCEKSGVKGLLSFEERKIE
CGSTPKDLLEAIVRALSIFSKDGIEGPYTLVINTDRWINFLKEEAGHYP
LEKRVEECLRGGKIITTPRIEDALVVSERGGDFKLILGQDLSIGYEDRE
KDAVRLFITETFTMLLKFGSGSGSVTTLSGLSGEQGPSGDMTTEEDSAT
HIKFSKRDEDGRELAGATMELRDSSGKTISTWISDGHVKDFYLYPGKYT
FVETAAPDGYEVATAITFTVNEQGQVTVNGEATKGDAHTGSSGS

In SEQ ID NO: 5, residues 1-214 are the encapsulin subunit sequence, which is C-terminally fused via a glycine-serine linker (residues 215-220) to spycatcher (residues 221-328). Additionally, the encapsulin sequence contains G53C and R94C substitutions, which form a non-natural disulfide bond that increases stabilization of the nanoparticle.

For production purposes, in some embodiments, the recombinant LASV GPC ectodomain linked to the nanoparticle subunit can include an N-terminal signal peptide that is cleaved during cellular processing. For example, the recombinant LASV GPC ectodomain protomer linked to the protein nanoparticle subunit can include a signal peptide at its N-terminus including, for example, a native LASV GPC signal peptide

The protein nanoparticles can be expressed in appropriate cells (e.g., HEK 293 Freestyle cells) and fusion proteins are secreted from the cells self-assembled into nanoparticles. The nanoparticles can be purified using known techniques, for example by a few different chromatography procedures, e.g. Mono Q (anion exchange) followed by size exclusion (SUPEROSE® 6) chromatography.

### Virus-Like Particles

In some embodiments, a virus-like particle (VLP) is provided that includes a disclosed recombinant LASV GPC ectodomain trimer. Typically such VLPs include a recombinant LASV GPC ectodomain trimer that is membrane anchored by a C-terminal transmembrane domain, for example the recombinant LASV GPC ectodomain protomers in the trimer each can be linked to a transmembrane domain and cytosolic tail from LASV GP2. VLPs lack the viral components that are required for virus replication and thus represent a highly attenuated, replication-incompetent form of a virus. However, the VLP can display a polypeptide (e.g., a recombinant LASV GPC ectodomain trimer) that is analogous to that expressed on infectious virus particles and can eliciting an immune response to LASV GPC when administered to a subject. Virus like particles and methods of their production are known, and viral proteins from several viruses are known to form VLPs, including human papillomavirus, HIV (Kang et al., Biol. Chem. 380: 353-64 (1999)), Semliki-Forest virus (Notka et al., Biol. Chem. 380: 341-52 (1999)), human polyomavirus (Goldmann et al., J. Virol. 73: 4465-9 (1999)), rotavirus (Jiang et al., Vaccine 17: 1005-13 (1999)), parvovirus (Casal, Biotechnology and Applied Biochemistry, Vol 29, Part 2, pp 141-150 (1999)), canine parvovirus (Hurtado et al., J. Virol. 70: 5422-9 (1996)), hepatitis E virus (Li et al., J. Virol. 71: 7207-13 (1997)), and Newcastle disease virus. The formation of such VLPs can be detected by any suitable technique. Examples of suitable techniques known in the art for detection of VLPs in a medium include, e.g., electron microscopy techniques, dynamic light scattering (DLS), selective chromatographic separation (e.g., ion exchange, hydrophobic interaction, and/or size exclusion chromatographic separation of the VLPs) and density gradient centrifugation.

### III. Polynucleotides and Expression

Polynucleotides encoding a protomer of any of the disclosed recombinant LASV GPC ectodomain trimers are also provided. These polynucleotides include DNA, cDNA and RNA sequences which encode the protomer, as well as vectors including the DNA, cDNA and RNA sequences, such as a DNA or RNA vector used for immunization. The genetic code to construct a variety of functionally equivalent nucleic acids, such as nucleic acids which differ in sequence but which encode the same protein sequence, or encode a conjugate or fusion protein including the nucleic acid sequence.

In several embodiments, the nucleic acid molecule encodes a precursor of the protomer, that, when expressed in an appropriate cell, is processed into a disclosed LASV GPC ectodomain protomer that can self-assemble into the corresponding recombinant LASV GPC ectodomain trimer. For example, the nucleic acid molecule can encode a recombinant LASV GPC ectodomain including a N-terminal signal sequence for entry into the cellular secretory system that is proteolytically cleaved in the during processing of the recombinant LASV GPC ectodomain in the cell.

In several embodiments, the nucleic acid molecule encodes a precursor LASV GPC polypeptide that, when expressed in an appropriate cell, is processed into a disclosed recombinant LASV GPC ectodomain protomer including GP1 and GP2 ectodomain polypeptides, wherein the recombinant LASV GPC ectodomain protomer includes the stabilizing modifications described herein, and optionally can be linked to a trimerization domain, such as a T4 Fibritin trimerization domain.

Exemplary nucleic acids can be prepared by cloning techniques. Examples of appropriate cloning and sequencing techniques, and instructions sufficient to direct persons of skill through many cloning exercises are known (see, e.g., Sambrook et al. (Molecular Cloning: A Laboratory Manual, 4^{th} ed, Cold Spring Harbor, New York, 2012) and Ausubel et al. (In Current Protocols in Molecular Biology, John Wiley & Sons, New York, through supplement 104, 2013).

Nucleic acids can also be prepared by amplification methods. Amplification methods include polymerase chain reaction (PCR), the ligase chain reaction (LCR), the transcription-based amplification system (TAS), the self-sustained sequence replication system (3SR). A wide variety of cloning methods, host cells, and in vitro amplification methodologies are well known to persons of skill.

The polynucleotides encoding a disclosed recombinant LASV GPC ectodomain protomer can include a recombinant DNA which is incorporated into a vector (such as an expression vector) into an autonomously replicating plasmid or virus or into the genomic DNA of a prokaryote or eukaryote, or which exists as a separate molecule (such as a cDNA) independent of other sequences. The nucleotides can be ribonucleotides, deoxyribonucleotides, or modified forms of either nucleotide. The term includes single and double forms of DNA.

Polynucleotide sequences encoding a disclosed recombinant LASV GPC ectodomain protomer can be operatively linked to expression control sequences. An expression control sequence operatively linked to a coding sequence is ligated such that expression of the coding sequence is achieved under conditions compatible with the expression control sequences. The expression control sequences include, but are not limited to, appropriate promoters, enhancers, transcription terminators, a start codon (i.e., ATG) in front of a protein-encoding gene, splicing signal for introns, maintenance of the correct reading frame of that gene to permit proper translation of mRNA, and stop codons.

DNA sequences encoding the disclosed recombinant S ectodomain protomer can be expressed in vitro by DNA transfer into a suitable host cell. The cell may be prokaryotic or eukaryotic. The term also includes any progeny of the subject host cell. It is understood that all progeny may not be identical to the parental cell since there may be mutations that occur during replication. Methods of stable transfer, meaning that the foreign DNA is continuously maintained in the host, are known in the art.

Hosts can include microbial, yeast, insect and mammalian organisms. Methods of expressing DNA sequences having eukaryotic or viral sequences in prokaryotes are well known in the art. Non-limiting examples of suitable host cells include bacteria, archea, insect, fungi (for example, yeast), plant, and animal cells (for example, mammalian cells, such as human). Exemplary cells of use include Escherichia coli, Bacillus subtilis, Saccharomyces cerevisiae, Salmonella typhimurium, SF9 cells, C129 cells, 293 cells, Neurospora, and immortalized mammalian myeloid and lymphoid cell lines. Techniques for the propagation of mammalian cells in culture are well-known (see, e.g., Helgason and Miller (Eds.), 2012, Basic Cell Culture Protocols (Methods in Molecular Biology), 4^{th} Ed., Humana Press). Examples of commonly used mammalian host cell lines are VERO and HeLa cells, CHO cells, and WI38, BHK, and COS cell lines, although cell lines may be used, such as cells designed to provide higher expression, desirable glycosylation patterns, or other features. In some embodiments, the host cells include HEK293 cells or derivatives thereof, such as GnTI^{-/-} cells (ATCC® No. CRL-3022), or HEK-293F cells.

Transformation of a host cell with recombinant DNA can be carried out by conventional techniques. Where the host is prokaryotic, such as, but not limited to, E. coli, competent cells which are capable of DNA uptake can be prepared from cells harvested after exponential growth phase and subsequently treated by the CaCl₂) method using standard procedures. Alternatively, MgCl₂ or RbCl can be used. Transformation can also be performed after forming a protoplast of the host cell if desired, or by electroporation.

When the host is a eukaryote, such methods of transfection of DNA as calcium phosphate coprecipitates, conventional mechanical procedures such as microinjection, electroporation, insertion of a plasmid encased in liposomes, or viral vectors can be used. Eukaryotic cells can also be co-transformed with polynucleotide sequences encoding a disclosed antigen, and a second foreign DNA molecule encoding a selectable phenotype, such as the herpes simplex thymidine kinase gene. Another method is to use a eukaryotic viral vector, such as simian virus 40 (SV40) or bovine papilloma virus, to transiently infect or transform eukaryotic cells and express the protein (see for example, Viral Expression Vectors, Springer press, Muzyczka ed., 2011). Appropriate expression systems such as plasmids and vectors of use in producing proteins in cells including higher eukaryotic cells such as the COS, CHO, HeLa and myeloma cell lines.

In one non-limiting example, a disclosed immunogen is expressed using the pVRC8400 vector (described in Barouch et al., J. Virol., 79, 8828-8834, 2005).

Modifications can be made to a nucleic acid encoding a disclosed recombinant LASV GPC ectodomain protomer without diminishing its biological activity. Some modifications can be made to facilitate the cloning, expression, or incorporation of the targeting molecule into a fusion protein. Such modifications are well known to those of skill in the art and include, for example, termination codons, a methionine added at the amino terminus to provide an initiation, site, additional amino acids placed on either terminus to create conveniently located restriction sites, or additional amino acids (such as poly His) to aid in purification steps.

In some embodiments, the disclosed recombinant LASV GPC ectodomain protomer can be expressed in cells under conditions where the recombinant LASV GPC ectodomain protomer self-assembles into trimers which are secreted from the cells into the cell media. In such embodiments, each recombinant LASV GPC ectodomain protomer contains a leader sequence (signal peptide) that causes the protein to enter the secretory system, where the signal peptide is cleaved and the protomers form a trimer, before being secreted in the cell media. The medium can be centrifuged and recombinant LASV GPC ectodomain trimer purified from the supernatant.

### IV. Viral Vectors

A nucleic acid molecule encoding a protomer of a disclosed recombinant LASV GPC ectodomain trimer can be included in a viral vector, for example, for expression of the immunogen in a host cell, or for immunization of a subject as disclosed herein. In some embodiments, the viral vectors are administered to a subject as part of a prime-boost vaccination. In several embodiments, the viral vectors are included in a vaccine, such as a primer vaccine or a booster vaccine for use in a prime-boost vaccination.

In several examples, the viral vector can be replication-competent. For example, the viral vector can have a mutation in the viral genome that does not inhibit viral replication in host cells. The viral vector also can be conditionally replication-competent. In other examples, the viral vector is replication-deficient in host cells.

A number of viral vectors have been constructed, that can be used to express the disclosed antigens, including polyoma, i.e., SV40 (Madzak et al., 1992, J. Gen. Virol., 73:15331536), adenovirus (Berkner, 1992, Cur. Top. Microbiol. Immunol., 158:39-6; Berliner et al., 1988, Bio Techniques, 6:616-629; Gorziglia et al., 1992, J. Virol., 66:4407-4412; Quantin et al., 1992, Proc. Natl. Acad. Sci. USA, 89:2581-2584; Rosenfeld et al., 1992, Cell, 68:143-155; Wilkinson et al., 1992, Nucl. Acids Res., 20:2233-2239; Stratford-Perricaudet et al., 1990, Hum. Gene Ther., 1:241-256), vaccinia virus (Mackett et al., 1992, Biotechnology, 24:495-499), adeno-associated virus (Muzyczka, 1992, Curr. Top. Microbiol. Immunol., 158:91-123; On et al., 1990, Gene, 89:279-282), herpes viruses including HSV and EBV (Margolskee, 1992, Curr. Top. Microbiol. Immunol., 158:67-90; Johnson et al., 1992, J. Virol., 66:29522965; Fink et al., 1992, Hum. Gene Ther. 3:11-19; Breakfield et al., 1987, Mol. Neurobiol., 1:337-371; Fresse et al., 1990, Biochem. Pharmacol., 40:2189-2199), Sindbis viruses (H. Herweijer et al., 1995, Human Gene Therapy 6:1161-1167; U.S. Pat. Nos. 5,091,309 and 5,2217,879), alphaviruses (S. Schlesinger, 1993, Trends Biotechnol. 11:18-22; I. Frolov et al., 1996, Proc. Natl. Acad. Sci. USA 93:11371-11377) and retroviruses of avian (Brandyopadhyay et al., 1984, Mol. Cell Biol., 4:749-754; Petropouplos et al., 1992, J. Virol., 66:3391-3397), murine (Miller, 1992, Curr. Top. Microbiol. Immunol., 158:1-24; Miller et al., 1985, Mol. Cell Biol., 5:431-437; Sorge et al., 1984, Mol. Cell Biol., 4:1730-1737; Mann et al., 1985, J. Virol., 54:401-407), and human origin (Page et al., 1990, J. Virol., 64:5370-5276; Buchschalcher et al., 1992, J. Virol., 66:2731-2739). Baculovirus (Autographa californica multinuclear polyhedrosis virus; AcMNPV) vectors are also known in the art, and may be obtained from commercial sources (such as PharMingen, San Diego, Calif.; Protein Sciences Corp., Meriden, Conn.; Stratagene, La Jolla, Calif.).

In several embodiments, the viral vector can include an adenoviral vector that expresses a protomer of a disclosed recombinant LASV GPC ectodomain trimer. Adenovirus from various origins, subtypes, or mixture of subtypes can be used as the source of the viral genome for the adenoviral vector. Non-human adenovirus (e.g., simian, chimpanzee, gorilla, avian, canine, ovine, or bovine adenoviruses) can be used to generate the adenoviral vector. For example, a simian adenovirus can be used as the source of the viral genome of the adenoviral vector. A simian adenovirus can be of serotype 1, 3, 7, 11, 16, 18, 19, 20, 27, 33, 38, 39, 48, 49, 50, or any other simian adenoviral serotype. A simian adenovirus can be referred to by using any suitable abbreviation known in the art, such as, for example, SV, SAdV, SAV or sAV. In some examples, a simian adenoviral vector is a simian adenoviral vector of serotype 3, 7, 11, 16, 18, 19, 20, 27, 33, 38, or 39. In one example, a chimpanzee serotype C Ad3 vector is used (see, e.g., Peruzzi et al., Vaccine, 27:1293-1300, 2009). Human adenovirus can be used as the source of the viral genome for the adenoviral vector. Human adenovirus can be of various subgroups or serotypes. For instance, an adenovirus can be of subgroup A (e.g., serotypes 12, 18, and 31), subgroup B (e.g., serotypes 3, 7, 11, 14, 16, 21, 34, 35, and 50), subgroup C (e.g., serotypes 1, 2, 5, and 6), subgroup D (e.g., serotypes 8, 9, 10, 13, 15, 17, 19, 20, 22, 23, 24, 25, 26, 27, 28, 29, 30, 32, 33, 36-39, and 42-48), subgroup E (e.g., serotype 4), subgroup F (e.g., serotypes 40 and 41), an unclassified serogroup (e.g., serotypes 49 and 51), or any other adenoviral serotype. Examples of replication-deficient adenoviral vectors, including multiply replication-deficient adenoviral vectors, are disclosed in U.S. Pat. Nos. 5,837,511; 5,851,806; 5,994,106; 6,127,175; 6,482,616; and 7,195,896, and International Patent Application Nos. WO 94/28152, WO 95/02697, WO 95/16772, WO 95/34671, WO 96/22378, WO 97/12986, WO 97/21826, and WO 03/022311.

### V. Immunogenic Compositions

Immunogenic compositions comprising a disclosed immunogen (e.g., recombinant LASV GPC ectodomain trimer) and a pharmaceutically acceptable carrier are also provided. Such compositions can be administered to subjects by a variety of administration modes, for example, intramuscular, subcutaneous, intravenous, intra-arterial, intra-articular, intraperitoneal, or parenteral routes. Methods for preparing administrable compositions are described in more detail in such publications as Remington: The Science and Practice of Pharmacy, 22^{nd} ed., London, UK: Pharmaceutical Press, 2013.

Thus, an immunogen described herein can be formulated with pharmaceutically acceptable carriers to help retain biological activity while also promoting increased stability during storage within an acceptable temperature range. Potential carriers include, but are not limited to, physiologically balanced culture medium, phosphate buffer saline solution, water, emulsions (e.g., oil/water or water/oil emulsions), various types of wetting agents, cryoprotective additives or stabilizers such as proteins, peptides or hydrolysates (e.g., albumin, gelatin), sugars (e.g., sucrose, lactose, sorbitol), amino acids (e.g., sodium glutamate), or other protective agents. The resulting aqueous solutions may be packaged for use as is or lyophilized. Lyophilized preparations are combined with a sterile solution prior to administration for either single or multiple dosing.

Formulated compositions, especially liquid formulations, may contain a bacteriostat to prevent or minimize degradation during storage, including but not limited to effective concentrations (usually ≤1% w/v) of benzyl alcohol, phenol, m-cresol, chlorobutanol, methylparaben, and/or propylparaben. A bacteriostat may be contraindicated for some patients; therefore, a lyophilized formulation may be reconstituted in a solution either containing or not containing such a component.

The immunogenic compositions of the disclosure can contain as pharmaceutically acceptable vehicles substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, and triethanolamine oleate.

The immunogenic composition may optionally include an adjuvant to enhance an immune response of the host. Suitable adjuvants are, for example, toll-like receptor agonists, alum, AlPO4, alhydrogel, Lipid-A and derivatives or variants thereof, oil-emulsions, saponins, neutral liposomes, liposomes containing the vaccine and cytokines, non-ionic block copolymers, and chemokines. Non-ionic block polymers containing polyoxyethylene (POE) and polyxylpropylene (POP), such as POE-POP-POE block copolymers, MPL™ (3-O-deacylated monophosphoryl lipid A; Corixa, Hamilton, IN) and IL-12 (Genetics Institute, Cambridge, MA), may be used as an adjuvant (Newman et al., 1998, Critical Reviews in Therapeutic Drug Carrier Systems 15:89-142). These adjuvants have the advantage in that they help to stimulate the immune system in a non-specific way, thus enhancing the immune response to a pharmaceutical product.

In some embodiments, the composition can be provided as a sterile composition. The pharmaceutical composition typically contains an effective amount of a disclosed immunogen and can be prepared by conventional techniques. Typically, the amount of immunogen in each dose of the immunogenic composition is selected as an amount which elicits an immune response without significant, adverse side effects. In some embodiments, the composition can be provided in unit dosage form for use to elicit an immune response in a subject, for example, to prevent LASV infection in the subject. A unit dosage form contains a suitable single preselected dosage for administration to a subject, or suitable marked or measured multiples of two or more preselected unit dosages, and/or a metering mechanism for administering the unit dose or multiples thereof. In other embodiments, the composition further includes an adjuvant.

### VI. Methods of Inducing an Immune Response

The disclosed immunogens (e.g., recombinant LASV GPC ectodomain trimer, a nucleic acid molecule (such as an RNA molecule) or vector encoding a protomer of a disclosed recombinant LASV GPC ectodomain trimer, or a protein nanoparticle or virus like particle comprising a disclosed recombinant LASV GPC ectodomain trimer) can be administered to a subject to induce an immune response to LASV GPC protein in the subject. In a particular example, the subject is a human. The immune response can be a protective immune response, for example a response that inhibits subsequent infection with LASV. Elicitation of the immune response can also be used to treat or inhibit LASV infection and illnesses associated with the LASV infection.

A subject can be selected for immunization that has or is at risk for developing LASV infection, for example because of exposure or the possibility of exposure to the LASV. Following administration of a disclosed immunogen, the subject can be monitored for infection or symptoms associated with LASV infection.

Typical subjects intended for immunization with the immunogens and methods of the present disclosure include humans, as well as non-human primates and other animals. To identify subjects for immunization according to the methods of the disclosure, accepted screening methods are employed to determine risk factors associated with a targeted or suspected disease or condition, or to determine the status of an existing disease or condition in a subject. These screening methods include, for example, conventional work-ups to determine environmental, familial, occupational, and other such risk factors that may be associated with the targeted or suspected disease or condition, as well as diagnostic methods, such as various ELISA and other immunoassay methods to detect and/or characterize LASV infection. These and other routine methods allow the clinician to select patients in need of immunization using the methods and pharmaceutical compositions of the disclosure.

The administration of a disclosed immunogen can be for prophylactic or therapeutic purpose. When provided prophylactically, the immunogen is provided in advance of any symptom, for example, in advance of infection. The prophylactic administration of the immunogen serves to prevent or ameliorate the course of any subsequent infection. When provided therapeutically, the immunogen is provided at or after the onset of a symptom of infection, for example, after development of a symptom of LASV infection or after diagnosis with the LASV infection. The immunogen can thus be provided prior to the anticipated exposure to the LASV so as to attenuate the anticipated severity, duration or extent of an infection and/or associated disease symptoms, after exposure or suspected exposure to the LASV, or after the actual initiation of an infection.

The immunogens described herein, and immunogenic compositions thereof, are provided to a subject in an amount effective to induce or enhance an immune response against the LASV GPC protein in the subject, preferably a human. The actual dosage of disclosed immunogen will vary according to factors such as the disease indication and particular status of the subject (for example, the subject's age, size, fitness, extent of symptoms, susceptibility factors, and the like), time and route of administration, other drugs or treatments being administered concurrently, as well as the specific pharmacology of the composition for eliciting the desired activity or biological response in the subject. Dosage regimens can be adjusted to provide an optimum prophylactic or therapeutic response.

An immunogenic composition including one or more of the disclosed immunogens can be used in coordinate (or prime-boost) vaccination protocols or combinatorial formulations. In certain embodiments, novel combinatorial immunogenic compositions and coordinate immunization protocols employ separate immunogens or formulations, each directed toward eliciting an anti-viral immune response, such as an immune response to LASV GPC protein. Separate immunogenic compositions that elicit the anti-viral immune response can be combined in a polyvalent immunogenic composition administered to a subject in a single immunization step, or they can be administered separately (in monovalent immunogenic compositions) in a coordinate (or prime-boost) immunization protocol.

There can be several boosts, and each boost can be a different disclosed immunogen. In some examples that the boost may be the same immunogen as another boost, or the prime. The prime and boost can be administered as a single dose or multiple doses, for example two doses, three doses, four doses, five doses, six doses or more can be administered to a subject over days, weeks or months. Multiple boosts can also be given, such one to five (e.g., 1, 2, 3, 4 or 5 boosts), or more. Different dosages can be used in a series of sequential immunizations. For example a relatively large dose in a primary immunization and then a boost with relatively smaller doses.

In some embodiments, the boost can be administered about two, about three to eight, or about four, weeks following the prime, or about several months after the prime. In some embodiments, the boost can be administered about 5, about 6, about 7, about 8, about 10, about 12, about 18, about 24, months after the prime, or more or less time after the prime. Periodic additional boosts can also be used at appropriate time points to enhance the subject's “immune memory.” The adequacy of the vaccination parameters chosen, e.g., formulation, dose, regimen and the like, can be determined by taking aliquots of serum from the subject and assaying antibody titers during the course of the immunization program. In addition, the clinical condition of the subject can be monitored for the desired effect, e.g., prevention of infection or improvement in disease state (e.g., reduction in viral load). If such monitoring indicates that vaccination is sub-optimal, the subject can be boosted with an additional dose of immunogenic composition, and the vaccination parameters can be modified in a fashion expected to potentiate the immune response.

In some embodiments, the prime-boost method can include DNA-primer and protein-boost vaccination protocol to a subject. The method can include two or more administrations of the nucleic acid molecule or the protein.

For protein therapeutics, typically, each human dose will comprise 1-1000 μg of protein, such as from about 1 μg to about 100 μg, for example, from about 1 μg to about 50 μg, such as about 1 μg, about 2 μg, about 5 μg, about 10 μg, about 15 μg, about 20 μg, about 25 μg, about 30 μg, about 40 μg, or about 50 μg.

The amount utilized in an immunogenic composition is selected based on the subject population (e.g., infant or elderly). An optimal amount for a particular composition can be ascertained by standard studies involving observation of antibody titers and other responses in subjects. It is understood that an effective amount of a disclosed immunogen, such as a disclosed recombinant LASV GPC ectodomain trimer, viral vector, or nucleic acid molecule, in a immunogenic composition, can include an amount that is ineffective at eliciting an immune response by administration of a single dose, but that is effective upon administration of multiple dosages, for example in a prime-boost administration protocol.

Upon administration of an immunogen of this disclosure, the immune system of the subject typically responds by producing antibodies specific for the LASV GPC ectodomain trimer included in the immunogen. Such a response signifies that an immunologically effective dose was delivered to the subject.

In some embodiments, the antibody response of a subject will be determined in the context of evaluating effective dosages/immunization protocols. In most instances it will be sufficient to assess the antibody titer in serum or plasma obtained from the subject. Decisions as to whether to administer booster inoculations and/or to change the amount of the therapeutic agent administered to the individual can be at least partially based on the antibody titer level. The antibody titer level can be based on, for example, an immunobinding assay which measures the concentration of antibodies in the serum which bind to an antigen including, for example, the recombinant LASV GPC ectodomain trimer included in the immunogen.

LASV infection does not need to be completely eliminated or reduced or prevented for the methods to be effective. For example, elicitation of an immune response to LASV with one or more of the disclosed immunogens can reduce or inhibit LASV infection by a desired amount, for example, by at least 10%, at least 20%, at least 50%, at least 60%, at least 70%, at least 80%, at least 90%, at least 95%, at least 98%, or even at least 100% (elimination or prevention of detectable infected cells), as compared to LASV infection in the absence of the immunogen. In additional examples, LASV replication can be reduced or inhibited by the disclosed methods. LASV replication does not need to be completely eliminated for the method to be effective. For example, the immune response elicited using one or more of the disclosed immunogens can reduce LASV replication by a desired amount, for example, by at least 10%, at least 20%, at least 50%, at least 60%, at least 70%, at least 80%, at least 90%, at least 95%, at least 98%, or even at least 100% (elimination or prevention of detectable LASV replication, as compared to LASV replication in the absence of the immune response.

In some embodiments, the disclosed immunogen is administered to the subject simultaneously with the administration of the adjuvant. In other embodiments, the disclosed immunogen is administered to the subject after the administration of the adjuvant and within a sufficient amount of time to induce the immune response.

One approach to administration of nucleic acids is direct immunization with plasmid DNA, such as with a mammalian expression plasmid. Immunization by nucleic acid constructs is well known in the art and taught, for example, in U.S. Pat. No. 5,643,578 (which describes methods of immunizing vertebrates by introducing DNA encoding a desired antigen to elicit a cell-mediated or a humoral response), and U.S. Pat. Nos. 5,593,972 and 5,817,637 (which describe operably linking a nucleic acid sequence encoding an antigen to regulatory sequences enabling expression). U.S. Pat. No. 5,880,103 describes several methods of delivery of nucleic acids encoding immunogenic peptides or other antigens to an organism. The methods include liposomal delivery of the nucleic acids (or of the synthetic peptides themselves), and immune-stimulating constructs, or ISCOMS™, negatively charged cage-like structures of 30-40 nm in size formed spontaneously on mixing cholesterol and Quil ATM (saponin). Protective immunity has been generated in a variety of experimental models of infection, including toxoplasmosis and Epstein-Barr virus-induced tumors, using ISCOMS™ as the delivery vehicle for antigens (Mowat and Donachie, Immunol. Today 12:383, 1991). Doses of antigen as low as 1 μg encapsulated in ISCOMS™ have been found to produce Class I mediated CTL responses (Takahashi et al., Nature 344:873, 1990).

In some embodiments, a plasmid DNA vaccine is used to express a disclosed immunogen in a subject. For example, a nucleic acid molecule encoding a disclosed immunogen can be administered to a subject to induce an immune response to the LASV GPC included in the immunogen. In some embodiments, the nucleic acid molecule can be included on a plasmid vector for DNA immunization, such as the pVRC8400 vector (described in Barouch et al., J. Virol, 79, 8828-8834, 2005).

In another approach to using nucleic acids for immunization, a disclosed recombinant LASV GPC ectodomain can be expressed by attenuated viral hosts or vectors or bacterial vectors. Recombinant vaccinia virus, adeno-associated virus (AAV), herpes virus, retrovirus, cytomegalo virus or other viral vectors can be used to express the peptide or protein, thereby eliciting a CTL response. For example, vaccinia vectors and methods useful in immunization protocols are described in U.S. Pat. No. 4,722,848. BCG (Bacillus Calmette Guerin) provides another vector for expression of the peptides (see Stover, Nature 351:456-460, 1991).

In one embodiment, a nucleic acid encoding a disclosed recombinant LASV GPC ectodomain trimer is introduced directly into cells. For example, the nucleic acid can be loaded onto gold microspheres by standard methods and introduced into the skin by a device such as Bio-Rad's HELIOS™ Gene Gun. The nucleic acids can be “naked,” consisting of plasmids under control of a strong promoter. Typically, the DNA is injected into muscle, although it can also be injected directly into other sites. Dosages for injection are usually around 0.5 μg/kg to about 50 mg/kg, and typically are about 0.005 mg/kg to about 5 mg/kg (see, e.g., U.S. Pat. No. 5,589,466).

In another embodiment, an mRNA-based immunization protocol can be used to deliver a nucleic acid encoding a protomer of the LASV GPC ectodomain trimer to elicit an immune response to LASV GPC. mRNA vaccines preclude safety concerns about DNA integration into the host genome and can be directly translated in the host cell cytoplasm. Moreover, cell-free, in vitro synthesis of RNA avoids the manufacturing complications associated with viral vectors.

In some embodiments, mRNA vaccination is achieved using mRNA encoding a LASV GPC ectodomain as described herein and formulated as a lipid nanoparticle according to known methods, such as those described in WO2021154763, US20210228707, WO2017070626 and US2019/0192646. See, also, Jackson et al., N Engl J Med., 383(20): 1920-1921, 2020. For example the mRNA component is a modified mRNA with 1-methylpseudouridine in place of uridine and a 7mG(5′)ppp(5′)N1mpNp cap. The mRNA sequence includes a 5′ untranslated region (UTR), the immunogen (LASV GPC open reading frame), a 3′ UTR, and a polyA tail. In some embodiments, the ORF sequence is codon optimized relative to native sequence for mRNA expression in a human and to increase stability. In several embodiments, the mRNA is formulated in a lipid nanoparticle; for example, comprising a PEG-modified lipid, a non-cationic lipid, a sterol, an ionizable lipid, or any combination thereof. In some embodiments, the lipid nanoparticle is composed of 50 mol % ionizable lipid ((2 hydroxyethyl)(6 oxo 6-(undecycloxy)hexyl)amino)octanoate, 10 mol % 1,2 distearoyl sn glycerol-3 phosphocholine (DSPC), 38.5 mol % cholesterol, and 1.5 mol % 1-monomethoxypolyethyleneglycol-2,3, dimyristylglycerol with polyethylene glycol of average molecular weight 2000 (PEG2000 DMG). The mRNA/lipid nanoparticle composition may be provided in any suitable carrier, such as a sterile liquid for injection at a concentration of 0.5 mg/mL in 20 mM trometamol (Tris) buffer containing 87 mg/mL sucrose and 10.7 mM sodium acetate, at pH 7.5 and with appropriate diluent.

Additional exemplary forms of RNA-based vaccination that can be used to deliver a nucleic acid encoding a LASV GPC ectodomain trimer as described herein include conventional non-amplifying mRNA immunization (see, e.g., Petsch et al., “Protective efficacy of in vitro synthesized, specific mRNA vaccines against influenza A virus infection,” Nature biotechnology, 30(12): 1210-6, 2012) and self-amplifying mRNA immunization (see, e.g., Geall et al., “Nonviral delivery of self-amplifying RNA vaccines,” PNAS, 109(36): 14604-14609, 2012; Magini et al., “Self-Amplifying mRNA Vaccines Expressing Multiple Conserved Influenza Antigens Confer Protection against Homologous and Heterosubtypic Viral Challenge,” PLOS One, 11(8):e0161193, 2016; and Brito et al., “Self-amplifying mRNA vaccines,” Adv Genet., 89:179-233, 2015). In another embodiment, a circular RNA (circRNA)-based immunization protocol can be used to deliver a nucleic acid encoding the LASV GPC ectodomain to elicit an immune response to the LASV. In contrast to linear RNA, circRNA is stable due to its covalently closed ring structure, which protects it from exonuclease-mediated degradation. Although circRNA lacks the essential elements for cap-dependent translation, it can be engineered to enable protein translation through internal ribosome entry site (IRES) or the m6A modification incorporated to its 5′ UTR region. (See, e.g., Wesselhoeft, P. S. Kowalski, D. G. Anderson, Engineering circular RNA for potent and stable translation in eukaryotic cells. Nat Commun. 9, 2629, 2018; Yang et al., Extensive translation of circular RNAs driven by N(6)-methyladenosine. Cell Res. 27, 626-641, 2017; Kristensen et al. The biogenesis, biology, and characterization or circular RNAs. Nat. Rev. Genetics, 20, 675-691, 2029).

In some embodiments, administration of a therapeutically effective amount of one or more of the disclosed immunogens to a subject induces a neutralizing antibody response in the subject. To assess neutralization activity, following immunization of a subject, serum can be collected from the subject at appropriate time points, frozen, and stored for neutralization testing. Methods to assay for neutralization activity include, but are not limited to, plaque reduction neutralization (PRNT) assays, microneutralization assays, flow cytometry based assays, single-cycle infection assays.

The following examples are provided to illustrate certain particular features and/or embodiments. These examples should not be construed to limit the disclosure to the particular features or embodiments described.

### EXAMPLES

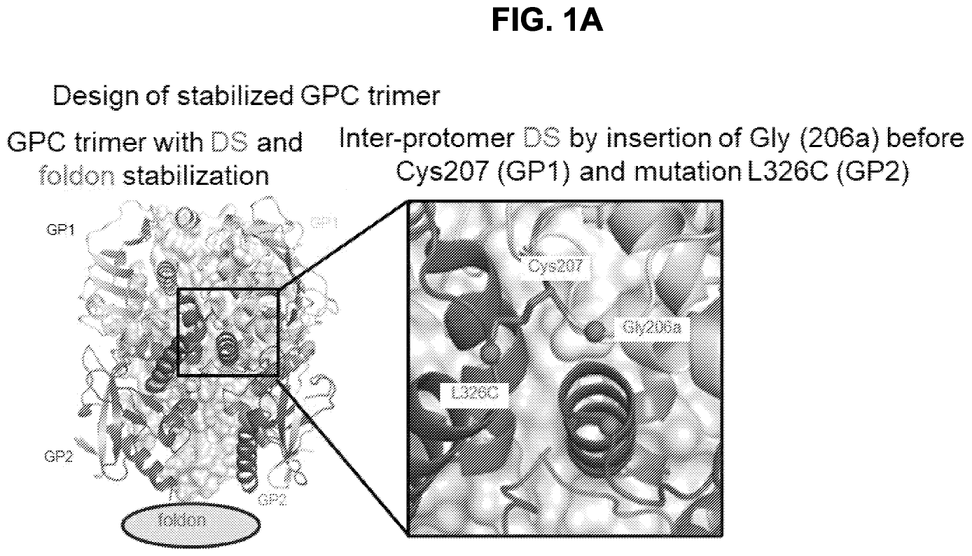

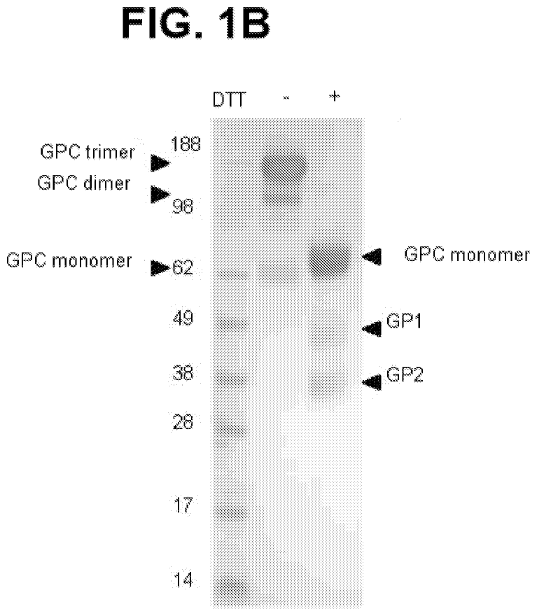

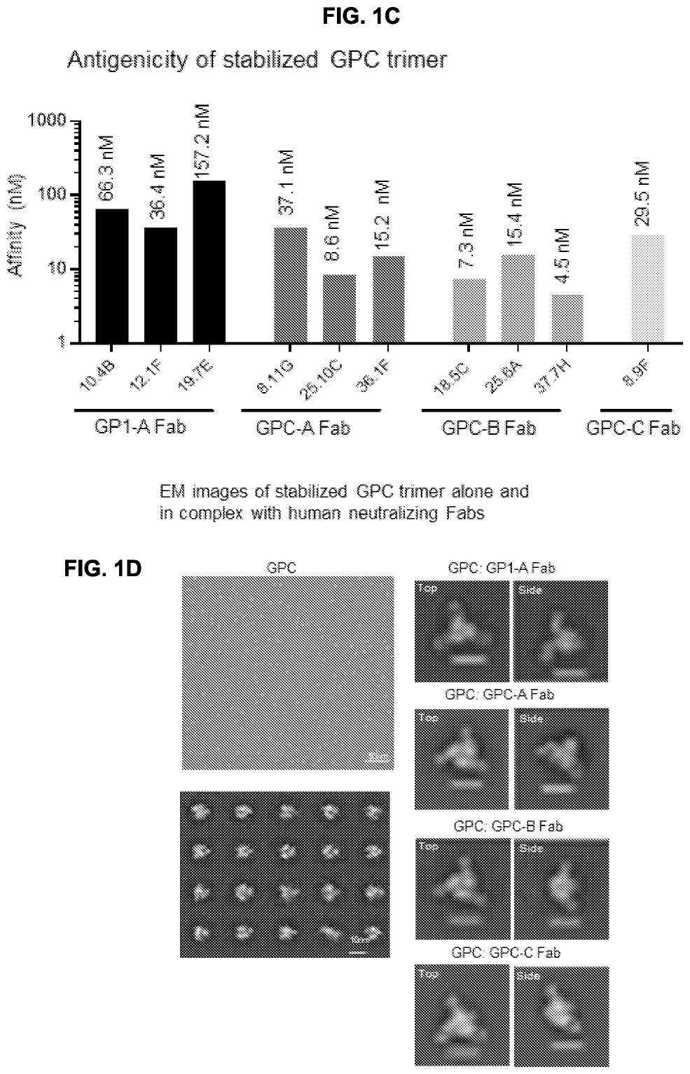

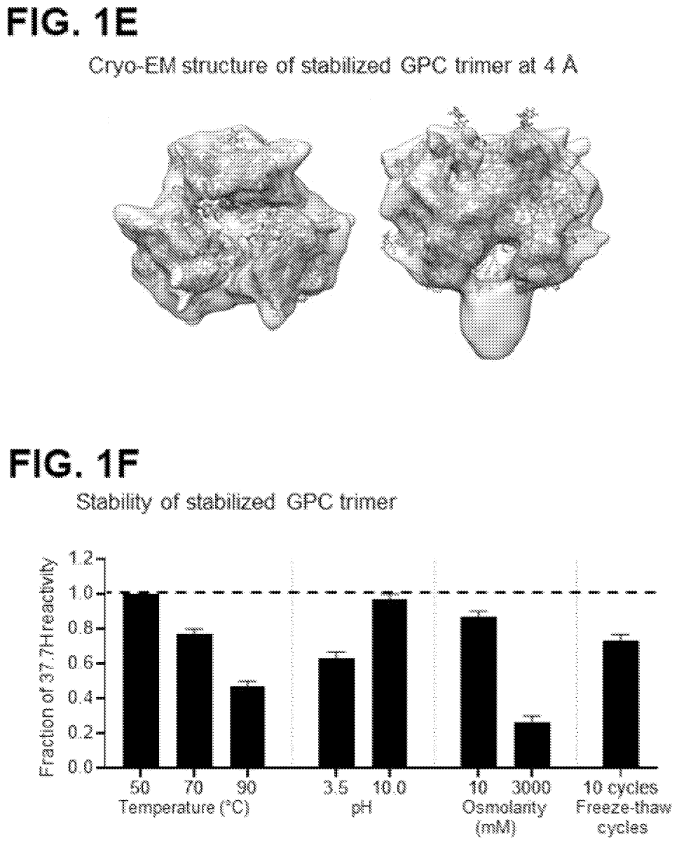

The LASV GPC trimer is metastable, conformationally labile, and heavily glycosylated, rendering the elicitation of neutralizing antibodies difficult (Cross et al., Curr Opin Virol 37, 97-104, 2019). Stabilization by a human neutralizing antibody 37.7H has enabled the structural analysis of the LASV GPC trimer (Hastie et al., Science 356, 923-928, 2017), yet a stabilized stand-alone GPC trimer has not been generated for use as an immunogen. As disclosed in the following Examples, a structure-based design strategy was employed to engineer an inter-protomer disulfide bond and a foldon trimerization domain to stabilize the soluble, ligand-free LASV GPC trimer in its prefusion state, which demonstrated similar antigenicity and trimeric architecture as the previously published antibody-bound LASV GPC structure (FIG. 1A).

### Example 1: Materials and Methods

This example describes the materials and experimental procedures for Example 2.

### Stabilized LASV GPC Trimer Sequence

The amino acid sequence of the stabilized LASV GPC trimer is provided below and set forth herein as residues 1-448 of SEQ ID NO: 2 (this includes the signal peptide, which is cleaved during cellular production).

MGQIVTFFQEVPHVIEEVMNIVLIALSVLAVLKGLYNFATCGLVGLVTF
LLLCGRSCTTSLYKGVYELQTLELNMETLNMTMPLSCTKNNSHHYIMVG
NETGLELTLTNTSIINHKFCNLSDAHKKNLYDHALMSIISTFHLSIPNF
NQYEAMSCDFNGGKISVQYNLSHSYAGDAANHCGTVANGVLQTFMRMAW
GGSYIALDSGGCGNWDCIMTSYQYLIIQNTTWEDHCQFSRPSPIGYLGL
LSQRTRDIYISRRRRGTFTWTLSDSEGKDTPGGYCLTRWMLIEAELKCF
GNTAVAKCNEKHDEEFCDMLRLFDFNKQAIQRCKAPAQMSIQLINKAVN
ALINDQLIMKNHLRDIMGIPYCNYSKYWYLNHTTTGRTSLPKCWLVSNG
SYLNETHFSDDIEQQADNMITEMLQKEGGGYIPEAPRDGQAYVRKDGEW
VLLSTFL

### Structure-Based Design of Stabilized LASV GPC Trimer

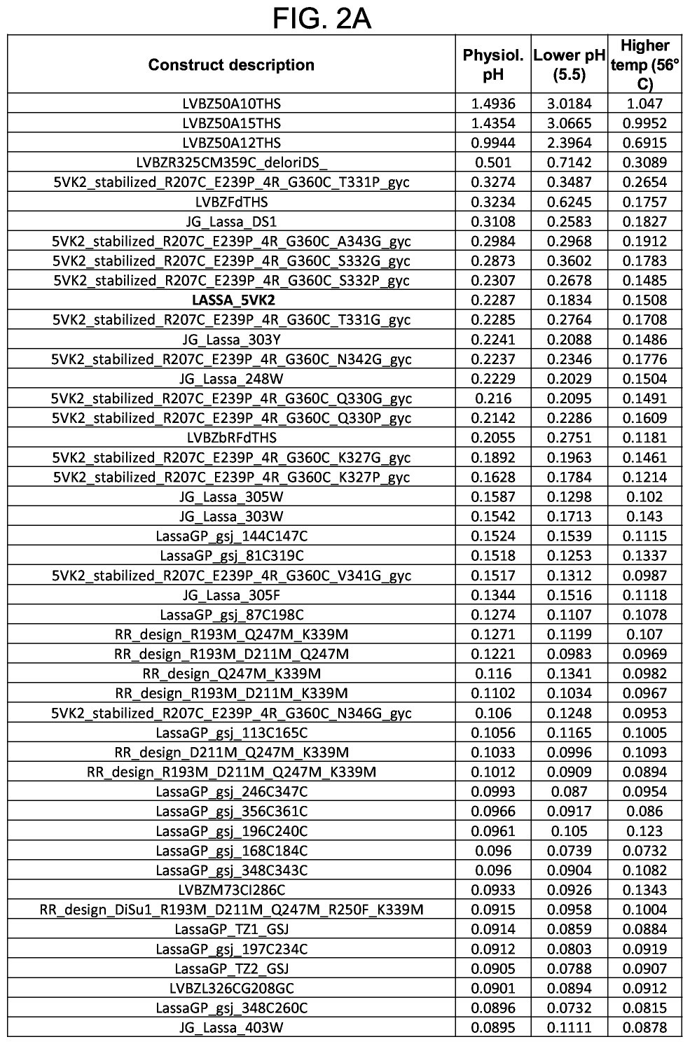

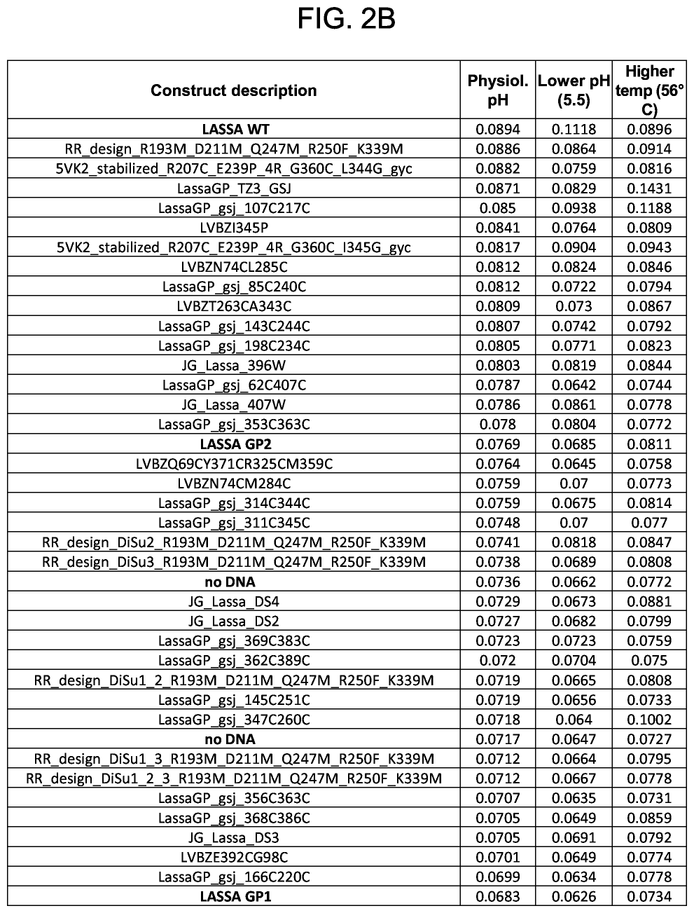

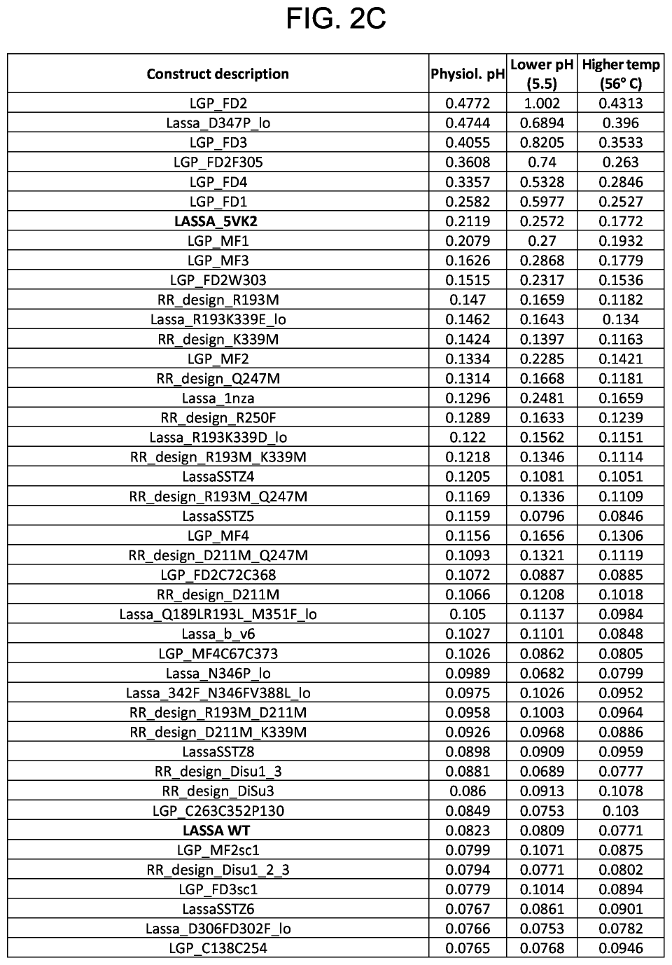

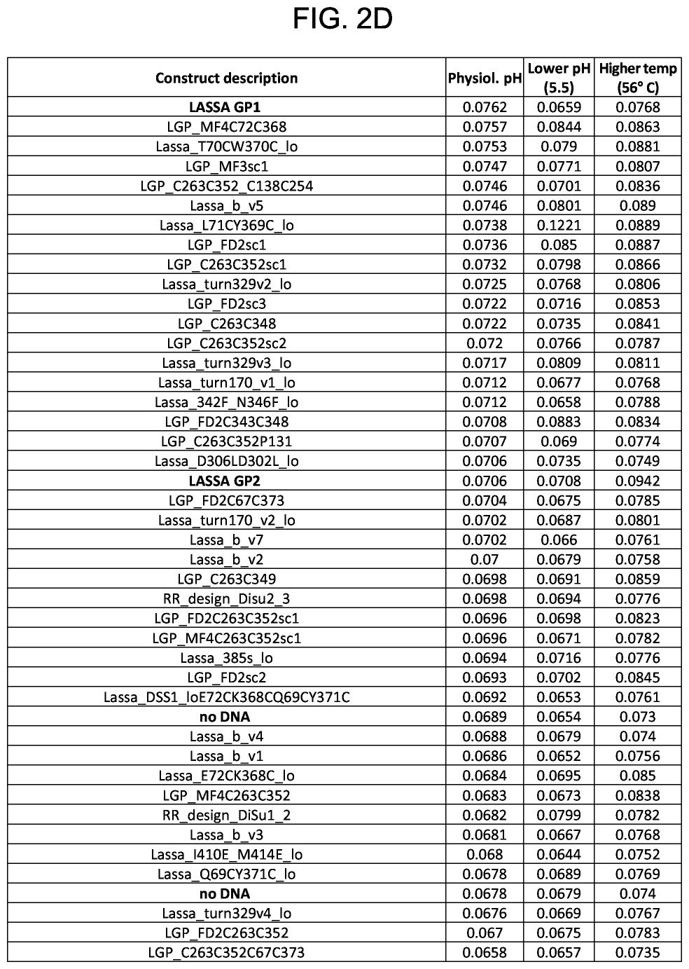

152 design variants based on the published LASV GPC-37.7H complex crystal structure (PDB ID 5VK2) (Hastie et al., Science 356, 923-928, 2017) were made. In total, 68 disulfide bonds, 43 cavity-filling mutations, 22 helix-breaking mutations, and 19 trimerization domain insertions were designed (FIGS. 2A and 2B).

### Antigenic Screening of LASV GPC Stabilizing Designs

Initial assessment of all constructs was performed using high-throughput 96-well microplate expression format followed by an ELISA-based antigenic evaluation as described previously (McLellan et al., Science 342, 592-598, 2013). Briefly, 2.5×10⁵ cells/ml of HEK 293T cells (Thermo Fisher Scientific, MA) were seeded in a 96-well microplate and cultured in expression medium (high glucose DMEM supplemented with 10% ultra-low IgG fetal bovine serum and 1×-non-essential amino acids) at 37° C., 5% CO₂ for 20 hours. Plasmid DNA and Turbo293 transfection reagent (Speed BioSystems) (Kwon et al., Cell Rep 22, 1798-1809, 2018) were then mixed and added to the cells. One day post transfection, enriched medium (high glucose DMEM plus 25% ultra-low IgG fetal bovine serum, 2× nonessential amino acids, 1× glutamine) was added to each well. The cells were cultured at 37° C. and 5% CO₂ for five additional days. Supernatants with the expressed LASV GPC variants were harvested and tested by ELISA for binding to 37.7H antibody using Ni²⁺-NTA microplates.

### Enzyme-Linked Immunosorbent Assay ELISA

Ni²⁺-NTA microplates (Pierce) were coated with 100 μl/well of the supernatant of LASV GPC variants at 4° C. overnight. After standard washing procedures with 0.1% Tween 20 in PBS, 100 μl of 10 μl/ml GPC-specific antibody 37.7H was added to the wells and incubated at room temperature for 2 hours. Next, 100 μl of horseradish peroxidase (HRP)-conjugated goat anti-human IgG Fc antibody (1:5000 v/v) (Jackson ImmunoResearch Laboratories Inc) was introduced to the plate for incubation at room temperature for 1 hour. Subsequently, plates were washed and then developed with 3,3′,5,5′-tetramethylbenzidine (TMB) substrate (Kirkegaard & Perry Laboratories). Signal was read at 450 nm by a plate reader (Beckman Coulter).

### LASV GPC Trimer Protein Expression and Purification

Disulfide and foldon-stabilized LASV GPC sequence was attached to a thrombin cleavage sequence, a hexahistidine tag, and a Strep-tag at its C-terminal end. The stabilized LASV GPC was expressed by transient transfection in 293F cells (Thermo Fisher) with Turbo293 transfection reagent (SPEED BioSystem) using the established protocol (Joyce et al., Cell 166, 609-623, 2016). Briefly, one liter of 293F cells at a density of 1.2×10⁶ cells/ml were co-transfected with 700 μg/liter of the LASV GPC expression plasmid and 300 μg/liter of furin plasmid. Six days post transfection, the culture supernatant was harvested and protein was purified from the supernatants by Nickel- (Roche) and Strep-Tactin- (IBA Lifesciences) affinity columns. The resultant protein was loaded on a Superdex 200 16/600 size exclusion column (GE Healthcare) to be further polished for use in subsequent assays.

### Production of Human LASV Antibodies

Immunoglobulin heavy chain or light chain sequences were constructed by gene synthesis and then cloned into human IgG1, lambda, or kappa expression plasmids as previously described (Tiller et al., J Immunol Methods 329, 112-124, 2008). Heavy and light chain expression plasmid DNA was transfected into Expi293F cells (Thermo Fisher) in 1:1 (v/v) ratio using Turbo293 transfection reagent (Kwon et al., Cell Rep 22, 1798-1809, 2018). Monoclonal antibodies from the culture supernatants were purified using recombinant Protein-A Sepharose (GE Healthcare) as per the manufacturer's instructions.

### Antibody Fab Preparation

The purified human IgG proteins were cleaved by LysC enzyme (1:4000 w/w) (Roche) at 37° C. overnight to yield Fabs. On the next day, the enzymatic digestion reaction was terminated by addition of protease inhibitor (Roche). The cleavage mixture was then passed through a Protein-A column to separate the Fc fragments from the Fab. The Fab collected in the flow-through was loaded onto a Superdex 200 16/60 column for further purification.

### LASV GPC Antigenic Characterization

An Octet Red384 instrument (fortéBio) was used to measure the binding kinetics between the stabilized LASV GPC trimers and human LASV neutralizing antibodies. Assays were performed at 30° C. in tilted black 384-well plates (Geiger Bio-One). Ni-NTA sensor tips (fortéBio) were used to capture the histidine-tagged stabilized LASV GPC trimer for 300 seconds. Then, the biosensor tips were equilibrated for 60 seconds in PBS before measurement of association with antigen-binding fragments (Fabs) in solution (6.25 nM to 400 nM) for 180 seconds. Subsequently, Fabs were allowed to dissociate for 300 seconds. Parallel correction to subtract systematic baseline drift was carried out by subtraction of the measurements recorded for a loaded sensor dipped in PBS. Data analysis and curve fitting were carried out using the Octet Data Analysis Software 9.0 (fortéBio). Experimental data were fitted with the binding equations describing a 1:1 interaction. Global analysis of the data sets assuming reversible binding (full dissociation) were carried out using nonlinear least-squares fitting allowing a single set of binding parameters to be obtained simultaneously for all of the concentrations used in each experiment.

### Negative-Stain Electron Microscopy

The protein was diluted with a buffer containing 10 mM HEPES, pH 7.0, and 150 mM NaCl to a concentration of 0.02 mg/ml and adsorbed to a freshly glow-discharged carbon-coated copper grid. The grid was washed with the same buffer, and the adsorbed protein molecules were negatively stained with 0.7% uranyl formate. Micrographs were collected at a nominal magnification of 100,000 using SerialEM (Mastronarde, J Struct Biol 152, 36-51, 2005) on a FEI T20 electron microscope equipped with a 2 k×2 k Eagle camera and operated at 200 kV. The pixel size was 0.22 nm. Particles were picked automatically using in-house written software and extracted into 100×100-pixel boxes. Reference-free 2D classifications were performed using Relion (Scheres, J Struct Biol 180, 519-530, 2012).

### Physical Stability of the Designed LASV GPC Trimer

To assess the physical stability of the designed LASV GPC trimer under various stress conditions, the proteins were treated with a variety of pharmaceutically relevant stresses such as extreme pH, high temperature, low and high osmolarity, and repeated freeze/thaw cycles while at a concentration of 50 μg/ml. The physical stability of treated LASV GPC trimer was evaluated by the preservation of binding to the GPC-specific antibody 37.7H. Temperature treatments were carried out by incubating the stabilized LASV GPC protein solutions at 50° C., 70° C. and 90° C. for 60 minutes in a PCR cycler with heated lid.

In pH treatments, the stabilized LASV GPC protein solution was adjusted to pH 3.5 and pH 10.0 with appropriate buffers for incubation at room temperature for 60 minutes and subsequently neutralized to pH 7.5. In osmolarity treatments, the stabilized LASV GPC protein solutions originally containing 150 mM NaCl were either diluted with 2.5 mM Tris buffer (pH 7.5) to an osmolarity of 10 mM NaCl or adjusted with 4.5 M MgCl₂ to a final concentration of 3.0 M MgCl₂. Protein solutions were incubated for 60 minutes at room temperature and then returned to 150 mM salt by adding 5.0 M NaCl or dilution with 2.5 mM Tris buffer, respectively, and concentrated to 50 μg/ml. The freeze/thaw treatment was carried out by repeatedly freezing the stabilized LASV GPC protein solutions in liquid nitrogen and thawing at 37° C. ten times. The degree of physical stability is reported as the ratio of steady state 37.7H antibody-binding level before and after stress treatment.

### Example 2: Structure-Based Design and Characterization of Stabilized LASV GPC Trimer

As a type I viral fusion machine, the GPC trimer is metastable and can readily change from the prefusion conformation to the more stable post-fusion conformation (Li et al., PLOS Pathog 12, e1005418, 2016; Willard et al., Pathogens 8(1):1, 2018). Since the epitopes for most neutralizing antibodies are present only in the prefusion conformation of the trimer, stabilization of this conformation of the trimer was pursued. Using the structure of the prefusion LASV GPC (GPCysR4) in complex with 37.7H Fab (PDB: 5VK2) (Hastie et al., Science 356, 923-928, 2017) as a template, over 150 variants were designed and screened for high binding affinity to the GPC-specific antibody 37.7H (FIG. 2). From these screening results, an engineered inter-protomer disulfide bond that links GP1 subunit of one protomer to the GP2 subunit of a neighboring protomer to yield improved antigenticity was identified. This inter-protomer disulfide, C207-L326C, replaced the existing intra-protomer disulfide C207-C360 present in GPCysR4 by introducing both a C360G mutation and a L326C mutation relative to GPCysR4. Residues 360 is glycine in WT sequence, thus the C360G substitution is a reversion to WT. Additionally, a Gly residue was inserted after position 206 (G206a) to allow the two Cys side chains to have optimal geometry for the formation of a 207C_{GP1}-L326C_{GP2} inter-protomer disulfide bond (FIG. 1A). To further stabilize the trimeric conformation of the LASV GPC, a T4-fibritin (foldon) trimerization domain was appended at the C-terminus to fix the protein base (FIG. 1A).

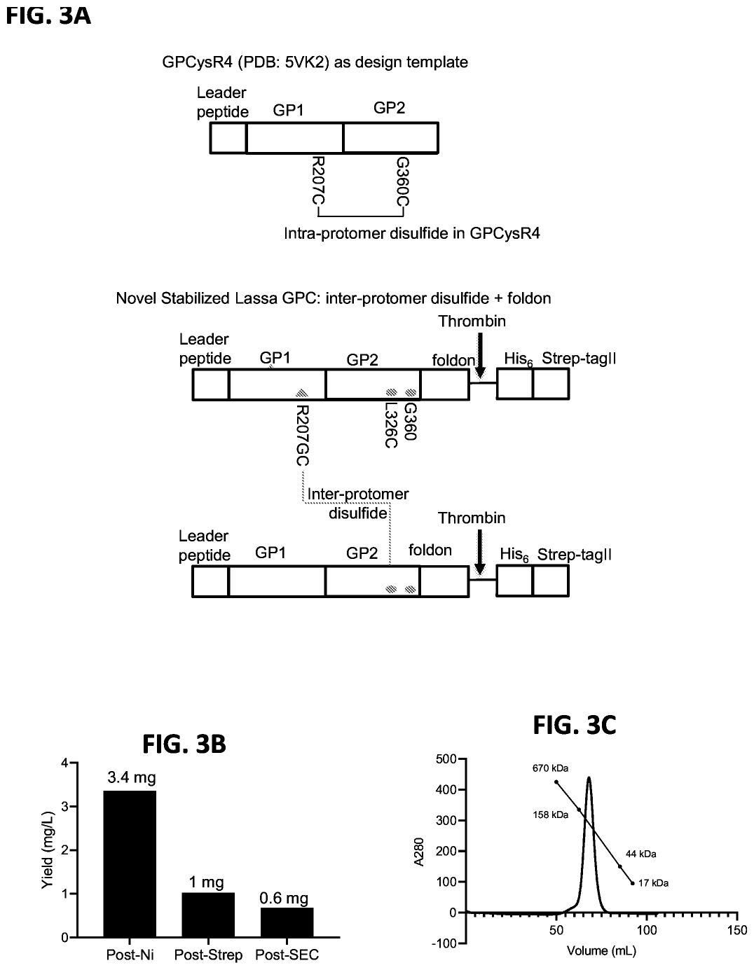

The resultant LASV GPC trimer expressed as a soluble protein with a final yield of approximately 0.5 mg/L by transient transfection of mammalian cells (FIGS. 3A-3C). The purified protein gave a major band at an expected size of a trimer (˜200 kDa) on SDS-PAGE in the absence of reducing agent, indicative of the formation of an inter-protomer disulfide bond (FIG. 1B). In the presence of reducing agent, bands for GP1 and GP2 appeared; however, the majority of the stabilized trimer existed as a band of the size for a protomer, indicating that GP1 and GP2 subunits were not efficiently cleaved (FIG. 1B). To verify the prefusion conformation of the stabilized LASV GPC trimer, its antigenicity was analyzed by bio-layer interferometry (BLI) assay for recognition by a panel of 10 human LASV neutralizing antibodies from four epitope groups; all except for the GPC-C antibody 8.9F could bind, suggesting that the stabilized trimer possessed generally similar antigenic properties as the previously published GPCysR4 construct (Hastie et al., Science 356, 923-928, 2017) (FIG. 1C). Negative-stain electron microscopy (EM) confirmed the homogeneous size and shape expected for a stable trimer in prefusion conformation of this stabilized GPC (FIG. 1D). In the presence of various human neutralizing Fabs, the stabilized trimer preserved its trimeric shape (FIG. 1D). To further examine the overall architecture of the inter-protomer disulfide-stabilized LASV GPC trimer, a cryo-EM structure was determined at 5.8 Å resolution from 47,597 particles (FIG. 1F). Trimers with C3 symmetry were observed, but the majority of the particles displayed CI symmetry. Lastly, the stabilized GPC trimer could withstand physical stresses under various temperature (50° C.-90° C.), pH (3.5 and 10), osmolarity (10 mM and 3000 mM NaCl), and freeze-thaw conditions, as evidenced by the retained 37.7H reactivity (Hastie et al., Science 356, 923-928, 2017) after treatments (FIG. 1D). Overall, the design yielded a soluble prefusion-stabilized LASV GPC, which exhibited desired antigenic and structural characteristics.

### Example 3: Design and Production of Encapsulin Nanopaticles Displaying Prefusion LASV GPC Ectodomain Trimer

This example describes design and production of an encapsulin nanoparticle linked to the prefusion LARV GPC trimer.

Protomers of the LASV GPC ectodomain trimer discussed above (residues 1-448 of SEQ ID NO: 2) were C-terminally fused to a spytag moiety. The sequence of the fusion protein is provided as SEQ ID NO: 2:

MGQIVTFFQEVPHVIEEVMNIVLIALSVLAVLKGLYNFATCGLVGLVTF
LLLCGRSCTTSLYKGVYELQTLELNMETLNMTMPLSCTKNNSHHYIMVG
NETGLELTLINTSIINHKFCNLSDAHKKNLYDHALMSIISTFHLSIPNF
NQYEAMSCDFNGGKISVQYNLSHSYAGDAANHCGTVANGVLQTFMRMAW
GGSYIALDSGGCGNWDCIMTSYQYLIIQNTTWEDHCQFSRPSPIGYLGL
LSQRTRDIYISRRRRGTFTWTLSDSEGKDTPGGYCLTRWMLIEAELKCF
GNTAVAKCNEKHDEEFCDMLRLFDFNKQAIQRCKAPAQMSIQLINKAVN
ALINDQLIMKNHLRDIMGIPYCNYSKYWYLNHTTTGRTSLPKCWLVSNG
SYLNETHFSDDIEQQADNMITEMLQKEGGGYIPEAPRDGQAYVRKDGEW
VLLSTFLGGGSGAHIVMVDAYKPTKGG

LASV GPC ectodomain trimer stabilized in the prefusion conformation and fused to spytag was expressed and purified as discussed above for the non-spytag version.

Additionally, encapsulin subunits were C-terminally fused to a spycatcher moiety. The sequence of the resulting fusion protein is provided as SEQ ID NO: 6:

MEFLKRSFAPLTEKQWQEIDNRAREIFKTQLYGRKFVDVEGGGGGHHHH
HHGGGGGPYGWEYAAHPLCEVEVLSDENEVVKWGLRKSLPLIELRATFT
LLWELDNLECGKPNVDLSSLEETVRKVAEFEDEVIFRGCEKSGVKGLLS
FEERKIECGSTPKDLLEAIVRALSIFSKDGIEGPYTLVINTDRWINFLK
EEAGHYPLEKRVEECLRGGKIITTPRIEDALVVSERGGDFKLILGQDLS
IGYEDREKDAVRLFITETFTMLLKFGSGSGSVTTLSGLSGEQGPSGDMT
TEEDSATHIKFSKRDEDGRELAGATMELRDSSGKTISTWISDGHVKDFY
LYPGKYTFVETAAPDGYEVATAITFTVNEQGQVTVNGEATKGDAHTGSS
GS

SEQ ID NO: 6 also includes an N-terminal signal peptide and His purification tag, and the encapsulin subunit includes G53C and R94C substitutions, which form a non-natural disulfide bond that increases stabilization of the nanoparticle.

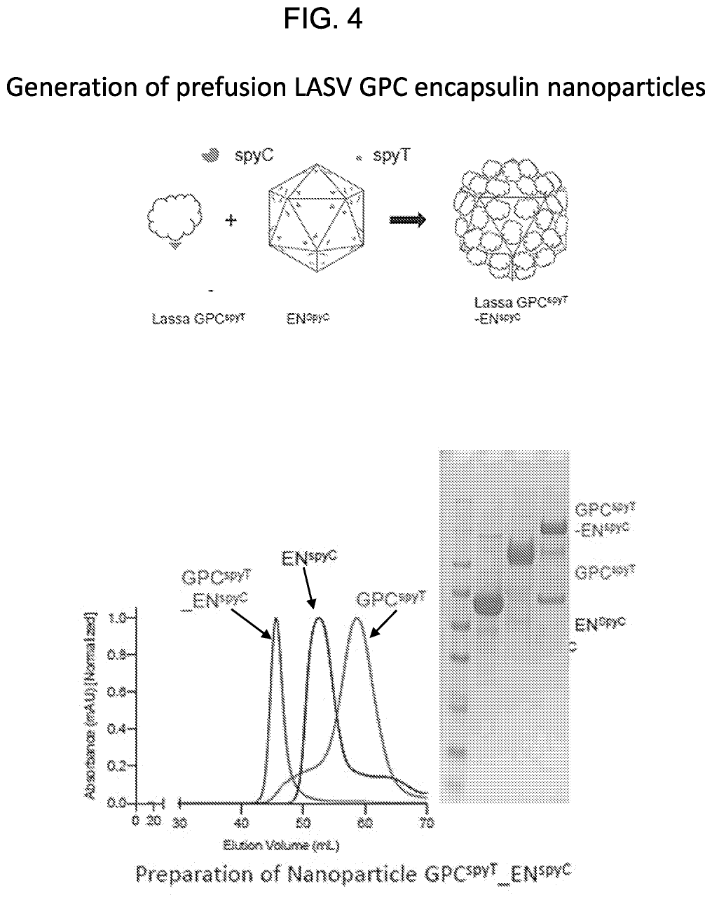

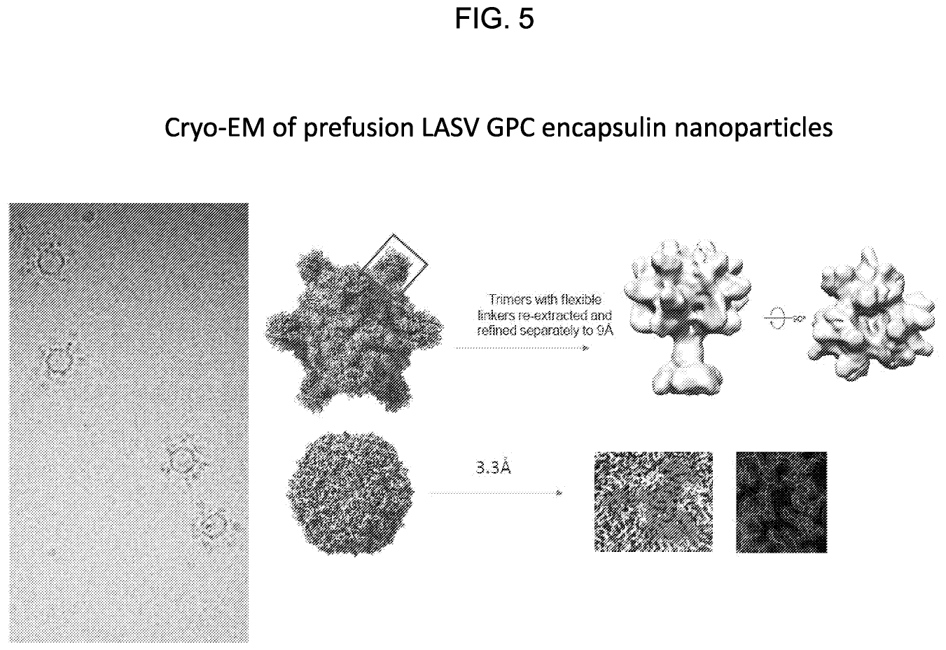

The encapsulin-spycatcher fusion was expressed in cells and the resulting self-assembled protein nanoparticles were harvested and incubated with the LASV GPC ectodomain trimer with spytag under conditions sufficient for formation of an isopeptide bond linking the trimer to the nanoparticle surface. The nanoparticles were purified by size-exclusion gel chromatography (See FIG. 4). Cyro-EM studies showed that the LASV GPC ectodomain trimer linked to the encapsulin nanoparticle is in the correct prefusion conformation (FIG. 5).

### Example 4: Immunogenicity of Prefusion LASV GPC Trimer and Nanoparticles Containing Same

This example presents ruslts of immunogenicity assays for purified prefusion LASV GPC trimer, and encapsulin nanoparticles containing same.

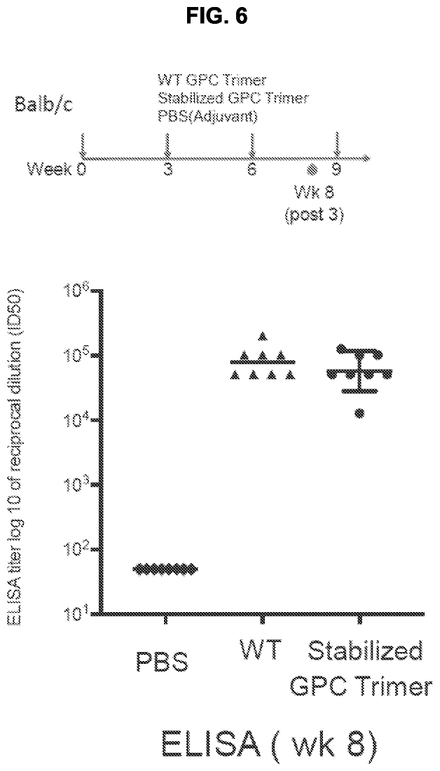

As depicted in FIG. 6, immunization assays using a mouse model were performed to show that the prefusion LASV GPC ectodomain trimer (Stabilized GPC Trimer in the figure), elicits an immune response targeting cognate antigen (prefusion LASV GPC ectodomain timer). ELISA analysis of immune sera harvested at week 8 following three immunizations of WT LASV GPC trimer or prefusion stabilized LASV GPC trimer (residues 59-448 of SEQ ID NO: 2) shows that each trimer elicited a substantial immune response.

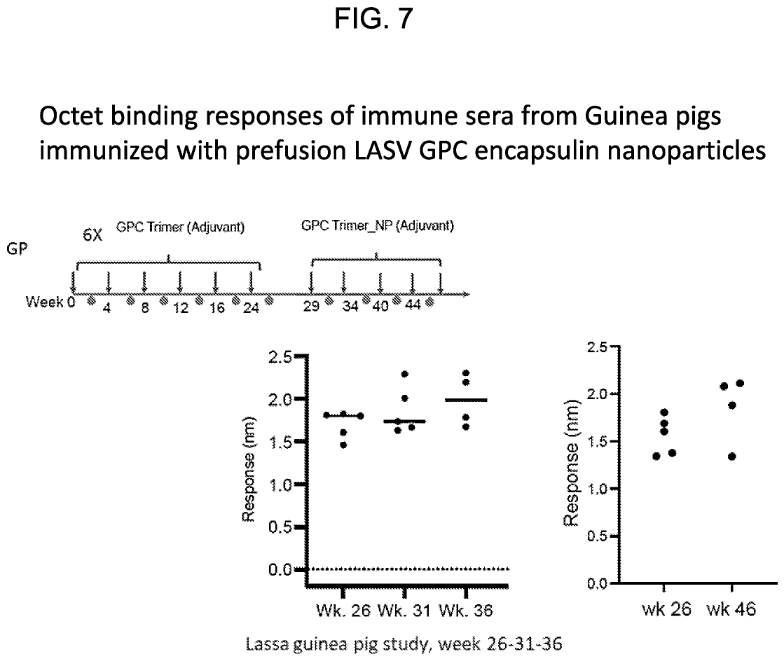

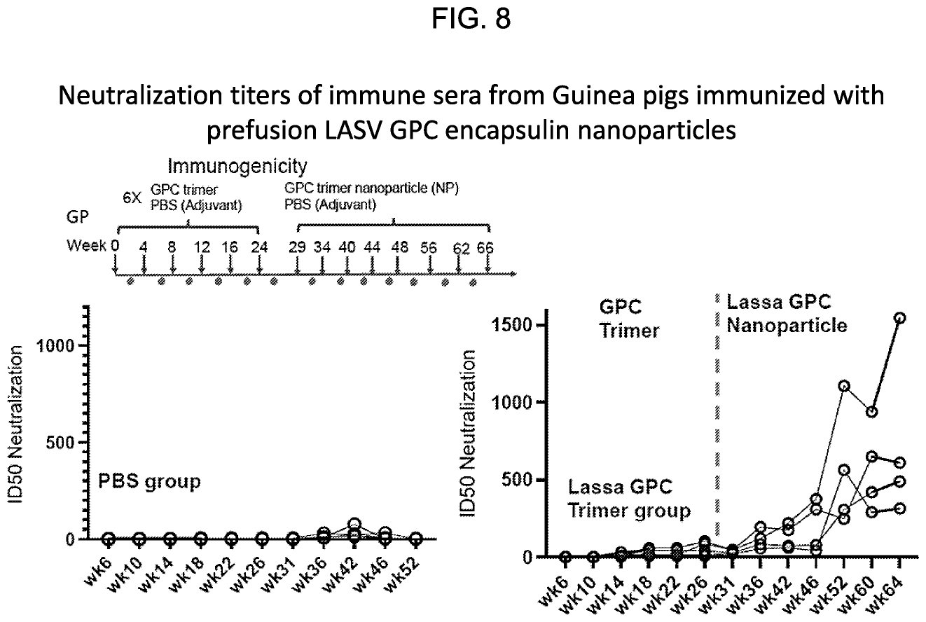

As depicted in FIG. 7, immunization assays using a guinea pig model were performed to show that the prefusion LASV GPC ectodomain trimer (residues 59-448 of SEQ ID NO: 2, GPC Trimer in the figure), as well as the encapsulin nanoparticle linked to the prefusion LASV GPC ectodomain trimer (GPC Trimer_NP in the figure), elicit an immune response targeting cognate antigen (prefusion LASV GPC ectodomain timer). FIG. 8 shows that the boost with encapsulin nanoparticle linked to the prefusion LASV GPC ectodomain trimer substantially incresed neutralizing titer compared to immune sera elicited from prefusion LASV GPC ectodomain trimer in non-nanoparticle format.

In view of the many possible embodiments to which the principles of the disclosure may be applied, it should be recognized that the illustrated embodiments are only examples and should not be taken as limiting the scope of the disclosure. Rather, the scope of the disclosure is defined by the following claims.

1. A recombinant Lassa virus (LASV) Glycoprotein Complex (GPC) ectodomain trimer stabilized in a prefusion conformation by amino acid substitutions in protomers of the trimer, wherein the amino acid substitutions comprise cysteine substitutions at GPC positions 207 and 326 that form a non-natural interprotomer disulfide bond, wherein the positioning of the amino acid substitutions is according to a reference LASV GPC protein sequence set forth as SEQ ID NO: 1.

2. The LASV GPC ectodomain trimer of claim 1, wherein the cysteine substitutions are R207GC and L326C substitutions.

3. (canceled)

4. The LASV GPC ectodomain trimer of claim 1, wherein the protomers comprise a GP1 protein comprising or consisting of GPC positions 59-259 and a GP2 ectodomain comprising or consisting of GPC positions 260-418.

5. The LASV GPC ectodomain trimer of claim 1, wherein:
the protomers further comprise amino acid substitutions to add arginine residues to a GP1/GP2 cleavage site at positions 256-259; or
the protomers further comprise a mutation to remove a GP1/GP2 cleavage site of the GPC ectodomain.

6. The LASV GPC ectodomain trimer of claim 5, wherein the protomers of the trimer comprise L258R and L259R substitutions.

7-8. (canceled)

9. The LASV GPC ectodomain trimer of claim 1, wherein:
the protomers of the trimer comprise an amino acid sequence at least 90% identical to residues 59-419 of SEQ ID NO: 2 and have the amino acid substitutions to stabilize the trimer in the prefusion conformation; or
the protomers of the timer comprise or consist of the amino acid sequence set forth as residues 59-419 of SEQ ID NO: 2.

10. (canceled)

11. The LASV GPC ectodomain trimer of claim 1, wherein the protomers of the trimer are fused C-terminally to a trimerization domain.

12. The LASV GPC ectodomain trimer of claim 11, wherein the trimerization domain is a T4 fibritin trimerization domain.

13. The LASV GPC ectodomain trimer of claim 11, wherein:
the protomers of the trimer fused to the trimerization domain comprise an amino acid sequence at least 90% identical to residues 59-448 of SEQ ID NO: 2 and have the amino acid substitutions to stabilize the trimer in the prefusion conformation; or
the protomers of the trimer fused to the trimerization domain comprise or consist of the amino acid sequence set forth as residues 59-448 of SEQ ID NO: 2.

14. (canceled)

15. The LASV GPC ectodomain trimer of claim 1, wherein the protomers of the trimer are fused C-terminally to a spytag moiety.

16. The LASV GPC ectodomain trimer of claim 15, wherein the protomers of the trimer fused to the spytag moiety comprise or consist of the amino acid sequence set forth as residues 59-468 of SEQ ID NO: 2.

17. The LASC GPC ectodomain trimer of claim 1, wherein the protomers of the trimer are linked to a heterologous protein.

18. The LASC GPC ectodomain trimer of claim 1, wherein the trimer is soluble.

19. The LASC GPC ectodomain trimer of claim 1, wherein the protomers of the LASV GPC ectodomain trimer are fused C-terminally to a transmembrane domain by a peptide linker, or directly fused to the transmembrane domain.

20. (canceled)

21. The LASC GPC ectodomain trimer of claim 1, conjugated to a heterologous carrier.

22. A protein nanoparticle comprising the LASC GPC ectodomain trimer of claim 1.

23. The protein nanoparticle of claim 22, wherein the LASC GPC ectodomain trimer is linked to subunits of a self-assembling protein nanoparticle.

24. The protein nanoparticle of claim 22, wherein:
the self-assembling protein nanoparticle is encapsuling; and/or
the LASC GPC ectodomain trimer is linked to the subunits of the self-assembling protein nanoparticle using a spytag/spycatcher system.

25. (canceled)

26. The protein nanoparticle of claim 24, wherein the protomers of the LASC GPC ectodomain trimer are C-terminally fused to a spytag moiety and the subunits of the self-assembling protein nanoparticle are linked to a spycatcher moiety.

27. The protein nanoparticle of claim 26, wherein the protomers of the trimer fused to the spytag moiety comprise or consist of the amino acid sequence set forth as residues 59-468 of SEQ ID NO: 2, and the encapsulin subunits fused to the spycatcher moiety comprise or consist of the amino acid sequence set forth as SEQ ID NO: 5.

28. A virus-like particle comprising the LASC GPC ectodomain trimer of claim 1.

29. A nucleic acid molecule encoding a protomer of the LASC GPC ectodomain trimer of claim 1.

30. The nucleic acid molecule of claim 29, operably linked to a promoter.

31. A vector comprising the nucleic acid molecule of claim 30.

32. The vector of claim 31, wherein the vector is an RNA vector.

33. A method of producing a LASC GPC ectodomain trimer, comprising:
expressing the nucleic acid molecule of claim 29, or a vector comprising the nucleic acid molecule, in a host cell; and
purifying the LASC GPC ectodomain trimer.

34. The LASC GPC ectodomain trimer produced by the method of claim 33.

35. An immunogenic composition, comprising the LASC GPC ectodomain trimer of claim 1, and a pharmaceutically acceptable carrier.

36. A method of eliciting an immune response to LASV GPC in a subject, comprising administering to the subject an effective amount of the immunogenic composition of claim 35 to elicit the immune response.

37. The method of claim 36, wherein the immune response inhibits LASV infection in the subject.
